SEQUENCE LISTING (12 sequences)

SEQ ID NO: 1 - protein, 444 aa, Artificial Sequence
  Heavy chain variable region
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 219 aa, Artificial Sequence
  Light chain variable region
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 15 aa, Artificial Sequence
  Pembrolizumab Light Chain
RASKGVSTSGYSYLH

SEQ ID NO: 4 - protein, 7 aa, Artificial Sequence
  Pembrolizumab Light Chain
LASYLES

SEQ ID NO: 5 - protein, 9 aa, Artificial Sequence
  Pembrolizumab Light Chain
QHSRDLPLT

SEQ ID NO: 6 - protein, 111 aa, Artificial Sequence [sequence omitted]

SEQ ID NO: 7 - protein, 218 aa, Artificial Sequence
  Pembrolizumab Light Chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 5 aa, Artificial Sequence
  Pembrolizumab Heavy Chain
NYYMY

SEQ ID NO: 9 - protein, 17 aa, Artificial Sequence
  Pembrolizumab Heavy Chain
GINPSNGGTNFNEKFKN

SEQ ID NO: 10 - protein, 11 aa, Artificial Sequence
  Pembrolizumab Heavy Chain
RDYRFDMGFDY

SEQ ID NO: 11 - protein, 120 aa, Artificial Sequence [sequence omitted]

SEQ ID NO: 12 - protein, 447 aa, Artificial Sequence
  Pembrolizumab Heavy Chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2023/0203175 A1
(43) Pub. Date: Jun. 29, 2023

(54) Anti-CD40 Antibody Combination Treatment for Cancer

(71) Applicants: SEAGEN INC., Bothell, WA (US); MSD INTERNATIONAL GMBH, Luzerne 6 (CH)

(72) Inventors: Michael Schmitt, Bothell, WA (US); Shyra Gardai, Bothell, WA (US)

(21) Appl. No.: 17/921,521

(22) PCT Filed: Apr. 27, 2021
(86) PCT No.: PCT/US2021/029315

§ 371 Date: Oct. 26, 2022

Related U.S. Application Data
(60) Provisional application No. 63/016,247, filed on Apr. 27, 2020.

(51) Int. Cl. C07K 16/28; A61K 31/337; A61P 35/00

(52) U.S. Cl. CPC C07K 16/2878; A61K 31/337; A61P 35/00; C07K 16/2896

(57) ABSTRACT

This disclosure relates to methods of treating cancer using a combination of an anti-CD40 antibody such as SEA-CD40, and an anti-PD-1 antibody such as pembrolizumab. The treatment can further include a chemotherapy.

### CLAIM OF PRIORITY

This application claims the benefit of U.S. Provisional Application No. 63/016,247, filed on Apr. 27, 2020. The entire contents of the foregoing are incorporated herein by reference.

### TECHNICAL FIELD

This disclosure relates to treatment of cancer using a combination of therapeutic agents.

### BACKGROUND

Treatment for cancer may involve administering more than one therapeutic agent. Various therapeutic agents have been tested either as single agent or as used in a combination therapy.

Anti-CD40 antibodies have been tested as potential therapeutics for treating cancer. CD40, a member of the tumor necrosis factor (TNF) receptor superfamily, is expressed on a variety of cell types including normal and neoplastic B cells, interdigitating cells, basal epithelial cells and carcinomas. The interaction of CD40 with its ligand/antigen, CD40L (also referred to as CD154, gp39, and TRAP), induces immune responses. A few anti-CD40 antibodies have been tested in clinical trials, but none has been approved by the FDA to date.

KEYTRUDA® (pembrolizumab), developed by Merck and Co., Inc., (Kenilworth NJ, USA) is an FDA-approved antibody therapeutic. To date, KEYTRUDA® has been approved for the treatment of various tumor and cancer types including certain melanoma, non-small cell lung cancer (NSCLC), small cell lung cancer (SCLC), head and neck squamous cell cancer (HNSCC), and classical Hodgkin lymphoma (cHL), among others. The prescription label of KEYTRUDA® is accessible at, e.g., FDA’s Approved Drug database.

Pembrolizumab, the active ingredient of KEYTRUDA®, is an anti-PD-1 antibody that binds to its ligand/antigen the Program Death receptor 1 (PD-1) and helps in the clearance of tumor cells by the immune system. PD-1 is an immunoglobulin superfamily member and negatively regulates antigen receptor signaling upon engagement of its ligands PD-L1 and/or PD-L2. Some cancers, however, are not responsive to anti-PD-1 or anti-PD-L1 treatments (Danaher P et al. J Immunother Cancer. 2018 Jun 22;6(1):63; Algazi et al. Cancer. 2016 Nov 15; 122(21): 3344-3353).

### BRIEF SUMMARY

A treatment regimen is described herein for treating cancer using a combination of an anti-CD40 antibody such as SEA-CD40, and pembrolizumab. A treatment regimen is also described herein for treating cancer using a combination of an anti-CD40 antibody such as SEA-CD40, pembrolizumab, and one or more chemotherapeutic agents. The one or more chemotherapeutic agent can include e.g., gemcitabine and/or paclitaxel (or Nab-paclitaxel). ABRAXANE® is a brand name of paclitaxel containing albumin-bound paclitaxel.

SEA-CD40 is a non-fucosylated or minimally fucosylated (“non-fucosylated” and “minimally fucosylated” are used interchangeably in this disclosure) anti-CD40 antibody, potently activates the innate immune system. SEA-CD40 is being tested as cancer treatment in clinical trial NCT02376699.

Methods of treating cancer using a combination of an anti-CD40 antibody, such as SEA-CD40, and pembrolizumab can benefit from synergistic effects. For example, SEA-CD40 can stimulate an initial innate immune response while blockade of the PD-1/PD-L1 axis can allow for a sustained, adaptive immune response.

Methods of treating cancer using a combination of an anti-CD40 antibody such as SEA-CD40 and pembrolizumab can further comprise administering one or more chemotherapeutic agents, e.g., gemcitabine and paclitaxel (or Nab-paclitaxel). ABRAXANE® is a brand name of paclitaxel containing albumin-bound paclitaxel.

In one aspect, this disclosure relates to a method of treating a pancreatic cancer, the method including administering to a patient having the pancreatic cancer: (i) a chemotherapy on day 1, day 8, and day 15 of each 28-day cycle, (ii) a composition comprising an anti-CD40 antibody on day 3 of each 28-day cycle, and (iii) an anti-PD-1 antibody on day 8 of each 42-day cycle.

In some embodiments, the anti-CD40 antibody comprises a heavy chain variable region comprising amino acid 1-113 of SEQ ID NO: 1 and a light chain variable region comprising amino acid 1-113 of SEQ ID NO: 2, and a human constant region; wherein the human constant region has an N-glycoside-linked sugar chain at residue N297 according to the EU index; and wherein less than 20% of N-glycoside-linked sugar chains in the composition comprise a fucose residue. In some embodiments, the anti-CD40 antibody is a SEA-CD40 variant.

In some embodiments, the anti-PD-1 antibody is pembrolizumab, nivolumab, h409A11, h409A16, h409A17, or AMP-514. In some embodiments, the anti-PD-1 antibody is Cemiplimab-rwlc, Spartalizumab, AK105, Tislelizumab, Dostarlimab, MEDI0680, Pidilizumab, AMP-224, or SHR-1210. In some embodiments, the anti-PD-1 antibody comprises a light chain comprising CDRs of SEQ ID NOs: 3-5, and a heavy chain comprising CDRs of SEQ ID NOs 8-10.

In some embodiments, less than 10% of N-glycoside-linked sugar chains in the composition comprising the anti-CD40 antibody has a fucose residue. In some embodiments, less than 5% of N-glycoside-linked sugar chains in the composition comprising the anti-CD40 antibody has a fucose residue. In some embodiments, less than 3% of N-glycoside-linked sugar chains in the composition comprising the anti-CD40 antibody has a fucose residue. In some embodiments, less than 2% of N-glycoside-linked sugar chains in the composition comprising the anti-CD40 antibody has a fucose residue.

In some embodiments, the anti-CD40 antibody comprises a heavy chain comprising the amino acid sequence of SEQ ID NO: 1 and a light chain comprising the amino acid sequence of SEQ ID NO: 2. In some embodiments, the anti-CD40 antibody is SEA-CD40. In some embodiments, the anti-CD40 antibody is a SEA-CD40 variant.

In some embodiments, the light chain of the anti-PD-1 antibody has a light chain variable region comprising the amino acid sequence of SEQ ID NO: 6, and the heavy chain of the anti-PD-1 antibody has a heavy chain variable region comprising the amino acid sequence of SEQ ID NO: 11. In some embodiments, the light chain of the anti-PD-1 antibody comprises the amino acid sequence of SEQ ID NO: 7, and the heavy chain of the anti-PD-1 antibody comprises the amino acid sequence of SEQ ID NO: 12. In some embodiments, the anti-PD-1 antibody is pembrolizumab. In some embodiments, the anti-PD-1 antibody is a pembrolizumab variant.

In some embodiments, the chemotherapy comprises gemcitabine and/or paclitaxel. In other words, the chemotherapy comprises gemcitabine, or paclitaxel, or both gemcitabine and paclitaxel. In some embodiments, paclitaxel is nab-paclitaxel. In some embodiments, paclitaxel is albumin-bound paclitaxel.

In some embodiments, the anti-CD40 antibody is administered at 10 µg/kg. In some embodiments, the anti-CD40 antibody is administered at 30 µg/kg. In some embodiments, the anti-PD-1 antibody is administered at 400 mg. In some embodiments, the anti-PD-1 antibody is administered intravenously.

In some embodiments, the pancreatic treated cancer is pancreatic ductal adenocarcinoma (PDAC).

In some embodiments, the anti-CD40 antibody is administered intravenously. In some embodiments, the anti-CD40 antibody is administered subcutaneously.

In another aspect, this disclosure relates to a method of treating a cancer, the method including: (i) administering a chemotherapy to a patient having the cancer in a cycle of every 4 weeks, (ii) administering a composition comprising an anti-CD40 antibody to the patient in a cycle of every 4 weeks, and (iii) administering an anti-PD-1 antibody to the patient in a cycle of every 3 weeks or 6 weeks. In some embodiments, the chemotherapy is administered on day 1, day 8, day 15 of each 4-week cycle, the anti-CD40 antibody is administered on day 3 of each 4-week cycle, and the anti-PD-1 antibody is administered on day 8 of each of the 3-week cycle or 6-week cycle.

In some embodiments, the anti-CD40 antibody comprises a heavy chain variable region comprising amino acid 1-113 of SEQ ID NO: 1 and a light chain variable region comprising amino acid 1-113 of SEQ ID NO: 2, and a human constant region; wherein the human constant region has an N-glycoside-linked sugar chain at residue N297 according to the EU index; and wherein less than 20% of N-glycoside-linked sugar chains in the composition comprise a fucose residue. In some embodiments, the anti-CD40 antibody is a SEA-CD40 variant.

In some embodiments, the anti-PD-1 antibody is pembrolizumab, nivolumab, h409A11, h409A16, h409A17, or AMP-514. In some embodiments, the anti-PD-1 antibody is Cemiplimab-rwlc, Spartalizumab, AK105, Tislelizumab, Dostarlimab, MEDI0680, Pidilizumab, AMP-224, or SHR-1210. In some embodiments, the anti-PD-1 antibody comprises a light chain comprising CDRs of SEQ ID NOs: 3-5, and a heavy chain comprising CDRs of SEQ ID NOs 8-10.

In some embodiments, less than 10% of N-glycoside-linked sugar chains in the composition comprising the anti-CD40 antibody has a fucose residue. In some embodiments, less than 5% of N-glycoside-linked sugar chains in the composition comprising the anti-CD40 antibody has a fucose residue. In some embodiments, less than 3% of N-glycoside-linked sugar chains in the composition comprising the anti-CD40 antibody has a fucose residue. In some embodiments, less than 2% of N-glycoside-linked sugar chains in the composition comprising the anti-CD40 antibody has a fucose residue.

In some embodiments, the anti-CD40 antibody comprises a heavy chain comprising the amino acid sequence of SEQ ID NO: 1 and a light chain comprising the amino acid sequence of SEQ ID NO: 2. In some embodiments, the anti-CD40 antibody is SEA-CD40. In some embodiments, the anti-CD40 antibody is a SEA-CD40 variant.

In some embodiments, the light chain of the anti-PD-1 antibody has a light chain variable region comprising the amino acid sequence of SEQ ID NO: 6, and the heavy chain of the anti-PD-1 antibody has a heavy chain variable region comprising the amino acid sequence of SEQ ID NO: 11. In some embodiments, the light chain of the anti-PD-1 antibody comprises the amino acid sequence of SEQ ID NO: 7, and the heavy chain of the anti-PD-1 antibody comprises the amino acid sequence of SEQ ID NO: 12. In some embodiments, the anti-PD-1 antibody is pembrolizumab. In some embodiments, the anti-PD-1 antibody is a pembrolizumab variant.

In some embodiments, the anti-PD-1 antibody is administered in a cycle of every 3 weeks, and the anti-PD-1 antibody is administered on day 8 of each 3-week cycle at a dose of 200 mg. In some embodiments, the anti-PD-1 antibody is administered in a cycle of every 6 weeks, and the anti-PD-1 antibody is administered on day 8 of each 6-week cycle at a dose of 400 mg. In some embodiments, the anti-PD-1 antibody is administered intravenously.

In some embodiments, the anti-CD40 antibody is administered at a dose of about 3 µg/kg, about 10 µg/kg, about 30 µg/kg, about 45 µg/kg, or about 60 µg/kg patient body weight. In some embodiments, the anti-CD40 antibody is administered at a dose of about 10 µg/kg patient body weight. In some embodiments, the anti-CD40 antibody is administered at a dose of about 30 µg/kg patient body weight.

In some embodiments, the cancer treated is melanoma; bladder cancer; lung cancer, e.g., small cell lung cancer and non-small cell lung cancer; ovarian cancer; kidney cancer; pancreatic cancer; breast cancer; cervical cancer; head and neck cancer, prostate cancer; glioblastoma; non-hodgkin lymphoma; chronic lymphocytic leukemia; hepatocellular carcinoma; or multiple myeloma. In some embodiments, the cancer treated is melanoma; breast cancer, metastatic breast cancer; lung cancer, non-small cell lung cancer (NSCLC), or pancreatic cancer. In some embodiments, the cancer treated is pancreatic cancer. In some embodiments, the cancer treated is pancreatic ductal adenocarcinoma (PDAC). In some embodiments, the cancer treated is metastatic pancreatic ductal adenocarcinoma.

In another aspect, this disclosure relates to a method of treating a cancer, the method including: (i) administering an anti-CD40 antibody to a patient having the cancer in a cycle of every week, every 2 weeks, every 3 weeks, every 4 weeks, every 5 weeks, every 6 weeks, every 7 weeks, or every 8 weeks, wherein the cycle includes a first cycle of administration of the anti-CD40 antibody, and (ii) administering an anti-PD-1 antibody to the patient in a cycle of every 3 weeks or every 6 weeks, wherein the cycle includes a first cycle of administration of the anti-PD-1 antibody. In some embodiments, a first administration of the anti-CD40 antibody in the first cycle of administration of the anti-CD40 antibody is 1 day, 2 days, 3 days, 4 days, 5 days, 6 days or 7 days prior to a first administration of the anti-PD-1 antibody in the first cycle of administration of the anti-PD-1 antibody.

In some embodiments, the anti-CD40 antibody comprises a heavy chain variable region comprising amino acid 1-113 of SEQ ID NO: 1 and a light chain variable region comprising amino acid 1-113 of SEQ ID NO: 2, and a human constant region; wherein the human constant region has an N-glycoside-linked sugar chain at residue N297 according to the EU index; and wherein less than 20% of N-glycoside-linked sugar chains in the composition comprise a fucose residue. In some embodiments, the anti-CD40 antibody is a SEA-CD40 variant.

In some embodiments, the anti-PD-1 antibody is pembrolizumab, nivolumab, h409A11, h409A16, h409A17, or AMP-514. In some embodiments, the anti-PD-1 antibody is Cemiplimab-rwlc, Spartalizumab, AK105, Tislelizumab, Dostarlimab, MEDI0680, Pidilizumab, AMP-224, or SHR-1210. In some embodiments, the anti-PD-1 antibody comprises a light chain comprising CDRs of SEQ ID NOs: 3-5, and a heavy chain comprising CDRs of SEQ ID NOs 8-10.

In some embodiments, less than 10% of N-glycoside-linked sugar chains in the composition comprising the anti-CD40 antibody has a fucose residue. In some embodiments, less than 5% of N-glycoside-linked sugar chains in the composition comprising the anti-CD40 antibody has a fucose residue. In some embodiments, less than 3% of N-glycoside-linked sugar chains in the composition comprising the anti-CD40 antibody has a fucose residue. In some embodiments, less than 2% of N-glycoside-linked sugar chains in the composition comprising the anti-CD40 antibody has a fucose residue.

In some embodiments, the anti-CD40 antibody comprises a heavy chain comprising the amino acid sequence of SEQ ID NO: 1 and a light chain comprising the amino acid sequence of SEQ ID NO: 2. In some embodiments, the anti-CD40 antibody is SEA-CD40. In some embodiments, the anti-CD40 antibody is a SEA-CD40 variant.

In some embodiments, the light chain of the anti-PD-1 antibody has a light chain variable region comprising the amino acid sequence of SEQ ID NO: 6, and the heavy chain of the anti-PD-1 antibody has a heavy chain variable region comprising the amino acid sequence of SEQ ID NO: 11. In some embodiments, the light chain of the anti-PD-1 antibody comprises the amino acid sequence of SEQ ID NO: 7, and the heavy chain of the anti-PD-1 antibody comprises the amino acid sequence of SEQ ID NO: 12. In some embodiments, the anti-PD-1 antibody is pembrolizumab. In some embodiments, the anti-PD-1 antibody is a pembrolizumab variant.

In some embodiments, the anti-CD40 antibody is administered in a cycle of every 2 weeks, every 4 weeks, every 6 weeks, or every 8 weeks. In some embodiments, the anti-CD40 antibody is administered in a cycle of every 4 weeks or every 8 weeks. In some embodiments, the anti-CD40 antibody is administered in a cycle of every 4 weeks.

In some embodiments, the anti-PD-1 antibody is administered in a cycle of every 3 weeks at a dose of 200 mg. In some embodiments, the anti-PD-1 antibody is administered in a cycle of every 6 weeks at a dose of 400 mg. In some embodiments, the anti-PD-1 antibody is administered intravenously.

In some embodiments, the first administration of the anti-CD40 antibody in the first cycle is 2 days, 3 days, 4 days, 5 days, or 6 days prior to the first administration of the anti-PD-1 antibody in the first cycle. In some embodiments, the first administration of the anti-CD40 antibody in the first cycle is 3 days, 4 days, or 5 days prior to the first administration of the anti-PD-1 antibody in the first cycle. In some embodiments, the first administration of the anti-CD40 antibody in the first cycle is 5 days prior to the first administration of the anti-PD-1 antibody in the first cycle.

In some embodiments, the anti-CD40 antibody and the anti-PD-1 antibody are administered in their first cycles according to a treatment regimen selected from the group consisting of: the anti-CD40 antibody is first administered on day 1, and the anti-PD-1 antibody is first administered on day 2; the anti-CD40 antibody is first administered on day 1, and the anti-PD-1 antibody is first administered on day 3; the anti-CD40 antibody is first administered on day 1, and the anti-PD-1 antibody is first administered on day 4; the anti-CD40 antibody is first administered on day 1, and the anti-PD-1 antibody is first administered on day 5; the anti-CD40 antibody is first administered on day 1, and the anti-PD-1 antibody is first administered on day 6; the anti-CD40 antibody is first administered on day 1, and the anti-PD-1 antibody is first administered on day 7; the anti-CD40 antibody is first administered on day 1, and the anti-PD-1 antibody is first administered on day 8; the anti-CD40 antibody is first administered on day 2, and the anti-PD-1 antibody is first administered on day 3; the anti-CD40 antibody is first administered on day 2, and the anti-PD-1 antibody is first administered on day 4; the anti-CD40 antibody is first administered on day 2, and the anti-PD-1 antibody is first administered on day 5; the anti-CD40 antibody is first administered on day 2, and the anti-PD-1 antibody is first administered on day 6; the anti-CD40 antibody is first administered on day 2, and the anti-PD-1 antibody is first administered on day 7; the anti-CD40 antibody is first administered on day 2, and the anti-PD-1 antibody is first administered on day 8; the anti-CD40 antibody is first administered on day 3, and the anti-PD-1 antibody is first administered on day 4; the anti-CD40 antibody is first administered on day 3, and the anti-PD-1 antibody is first administered on day 5; the anti-CD40 antibody is first administered on day 3, and the anti-PD-1 antibody is first administered on day 6; the anti-CD40 antibody is first administered on day 3, and the anti-PD-1 antibody is first administered on day 7; the anti-CD40 antibody is first administered on day 3, and the anti-PD-1 antibody is first administered on day 8; the anti-CD40 antibody is first administered on day 4, and the anti-PD-1 antibody is first administered on day 5; the anti-CD40 antibody is first administered on day 4, and the anti-PD-1 antibody is first administered on day 6; the anti-CD40 antibody is first administered on day 4, and the anti-PD-1 antibody is first administered on day 7; the anti-CD40 antibody is first administered on day 4, and the anti-PD-1 antibody is first administered on day 8; the anti-CD40 antibody is first administered on day 5, and the anti-PD-1 antibody is first administered on day 6; the anti-CD40 antibody is first administered on day 5, and the anti-PD-1 antibody is first administered on day 7; the anti-CD40 antibody is first administered on day 5, and the anti-PD-1 antibody is first administered on day 8; the anti-CD40 antibody is first administered on day 6, and the anti-PD-1 antibody is first administered on day 7; the anti-CD40 antibody is first administered on day 6, and the anti-PD-1 antibody is first administered on day 8; and the anti-CD40 antibody is first administered on day 7, and the anti-PD-1 antibody is first administered on day 8.

In some embodiments, the anti-CD40 antibody is first administered on day 1, and the anti-PD-1 antibody is first administered on day 3. In some embodiments, the anti-CD40 antibody is first administered on day 1, and the anti-PD-1 antibody is first administered on day 5. In some embodiments, the anti-CD40 antibody is first administered on day 1, and the anti-PD-1 antibody is first administered on day 8. In some embodiments, the anti-CD40 antibody is first administered on day 3, and the anti-PD-1 antibody is first administered on day 5. In some embodiments, the anti-CD40 antibody is first administered on day 3, and the anti-PD-1 antibody is first administered on day 8. In some embodiments, the anti-CD40 antibody is first administered on day 5, and the anti-PD-1 antibody is first administered on day 8.

In some embodiments, the anti-CD40 antibody is administered at a dose of about 3 µg/kg, about 10 µg/kg, about 30 µg/kg, about 45 µg/kg, or about 60 µg/kg patient body weight. In some embodiments, the anti-CD40 antibody is administered at a dose of about 10 µg/kg patient body weight. In some embodiments, the anti-CD40 antibody is administered at a dose of about 30 µg/kg patient body weight.

In some embodiments, the cancer treated is melanoma; bladder cancer; lung cancer, e.g., small cell lung cancer and non-small cell lung cancer; ovarian cancer; kidney cancer; pancreatic cancer; breast cancer; cervical cancer; head and neck cancer, prostate cancer; glioblastoma; non-hodgkin lymphoma; chronic lymphocytic leukemia; hepatocellular carcinoma; or multiple myeloma. In some embodiments, the cancer treated is melanoma; breast cancer, metastatic breast cancer; lung cancer, non-small cell lung cancer (NSCLC), or pancreatic cancer. In some embodiments, the cancer treated is pancreatic cancer. In some embodiments, the cancer treated is pancreatic ductal adenocarcinoma (PDAC). In some embodiments, the cancer treated is metastatic pancreatic ductal adenocarcinoma.

In another aspect, this disclosure relates to a method of treating a cancer, the method: (i) administering a chemotherapy to a patient having the cancer in a cycle of every 3 weeks, every 4 weeks, every 5 weeks, or every 6 weeks, (ii) administering an anti-CD40 antibody to a patient having the cancer in a cycle of every week, every 2 weeks, every 3 weeks, every 4 weeks, every 5 weeks, every 6 weeks, every 7 weeks, or every 8 weeks, and (iii) administering an anti-PD-1 antibody to the patient in a cycle of every 3 weeks or every 6 weeks. In some embodiments, a first administration of the chemotherapy in the first cycle of administration of the chemotherapy is 1 day, 2 days, 3 days, 4 days, 5 days, 6 days or 7 days prior to a first administration of the anti-CD40 antibody in the first cycle of administration of the anti-CD40 antibody. In some embodiments, a first administration of the anti-CD40 antibody in the first cycle of administration of the anti-CD40 antibody is 1 day, 2 days, 3 days, 4 days, 5 days, 6 days or 7 days prior to the first administration of the anti-PD-1 antibody in the first cycle of administration of the anti-PD-1 antibody.

In some embodiments, the anti-CD40 antibody comprises a heavy chain variable region comprising amino acid 1-113 of SEQ ID NO: 1 and a light chain variable region comprising amino acid 1-113 of SEQ ID NO: 2, and a human constant region; wherein the human constant region has an N-glycoside-linked sugar chain at residue N297 according to the EU index; and wherein less than 20% of N-glycoside-linked sugar chains in the composition comprise a fucose residue. In some embodiments, the anti-CD40 antibody is a SEA-CD40 variant.

In some embodiments, the anti-PD-1 antibody is pembrolizumab, nivolumab, h409A11, h409A16, h409A17, or AMP-514. In some embodiments, the anti-PD-1 antibody is Cemiplimab-rwlc, Spartalizumab, AK105, Tislelizumab, Dostarlimab, MEDI0680, Pidilizumab, AMP-224, or SHR-1210. In some embodiments, the anti-PD-1 antibody comprises a light chain comprising CDRs of SEQ ID NOs: 3-5, and a heavy chain comprising CDRs of SEQ ID NOs 8-10.

In some embodiments, less than 10% of N-glycoside-linked sugar chains in the composition comprising the anti-CD40 antibody has a fucose residue. In some embodiments, less than 5% of N-glycoside-linked sugar chains in the composition comprising the anti-CD40 antibody has a fucose residue. In some embodiments, less than 3% of N-glycoside-linked sugar chains in the composition comprising the anti-CD40 antibody has a fucose residue. In some embodiments, less than 2% of N-glycoside-linked sugar chains in the composition comprising the anti-CD40 antibody has a fucose residue.

In some embodiments, the anti-CD40 antibody comprises a heavy chain comprising the amino acid sequence of SEQ ID NO: 1 and a light chain comprising the amino acid sequence of SEQ ID NO: 2. In some embodiments, the anti-CD40 antibody is SEA-CD40. In some embodiments, the anti-CD40 antibody is a SEA-CD40 variant.

In some embodiments, the light chain of the anti-PD-1 antibody has a light chain variable region comprising the amino acid sequence of SEQ ID NO: 6, and the heavy chain of the anti-PD-1 antibody has a heavy chain variable region comprising the amino acid sequence of SEQ ID NO: 11. In some embodiments, the light chain of the anti-PD-1 antibody comprises the amino acid sequence of SEQ ID NO: 7, and the heavy chain of the anti-PD-1 antibody comprises the amino acid sequence of SEQ ID NO: 12. In some embodiments, the anti-PD-1 antibody is pembrolizumab. In some embodiments, the anti-PD-1 antibody is a pembrolizumab variant.

In some embodiments, the chemotherapy comprises one or both of gemcitabine and paclitaxel. In some embodiments, the chemotherapy comprises both gemcitabine and paclitaxel. In some embodiments, the chemotherapy consists of gemcitabine and paclitaxel. In some embodiments, paclitaxel is nab-paclitaxel. In some embodiments, paclitaxel is albumin-bound paclitaxel.

In some embodiments, the anti-CD40 antibody is administered in a cycle of every 2 weeks, every 4 weeks, every 6 weeks, or every 8 weeks. In some embodiments, the anti-CD40 antibody is administered in a cycle of every 4 weeks or every 8 weeks. In some embodiments, the anti-CD40 antibody is administered in a cycle of every 4 weeks. In some embodiments, the anti-PD-1 antibody is administered in a cycle of every 3 weeks at a dose of 200 mg. In some embodiments, the anti-PD-1 antibody is administered in a cycle of every 6 weeks at a dose of 400 mg. In some embodiments, the anti-PD-1 antibody is administered intravenously.

In some embodiments, the first administration of the chemotherapy in the first cycle is 2 days, 3 days, 4 days, 5 days, or 6 days prior to the first administration of the anti-CD40 antibody in the first cycle, and the first administration of the anti-CD40 antibody in the first cycle is 2 days, 3 days, 4 days, 5 days, or 6 days prior to the first administration of the anti-PD-1 antibody in the first cycle. In some embodiments, the first administration of the chemotherapy in the first cycle is 2 days, 3 days, or 4 days prior to the first administration of the anti-CD40 antibody in the first cycle, and the first administration of the anti-CD40 antibody in the first cycle is 3 days, 4 days, or 5 days prior to the first administration of the anti-PD-1 antibody in the first cycle. In some embodiments, the first administration of the chemotherapy in the first cycle is 2 days prior to the first administration of the anti-CD40 antibody in the first cycle, and the first administration of the anti-CD40 antibody in the first cycle is 5 days prior to the first administration of the anti-PD-1 antibody in the first cycle.

In some embodiments, the chemotherapy, the anti-CD40 antibody and the anti-PD-1 antibody are administered in their first cycles according to a treatment regimen selected from the group consisting of: the chemotherapy is first administered on day 1, the anti-CD40 antibody is first administered on day 2, and the anti-PD-1 antibody is first administered on day 3; the chemotherapy is first administered on day 1, the anti-CD40 antibody is first administered on day 2, and the anti-PD-1 antibody is first administered on day 4; the chemotherapy is first administered on day 1, the anti-CD40 antibody is first administered on day 2, and the anti-PD-1 antibody is first administered on day 5; the chemotherapy is first administered on day 1, the anti-CD40 antibody is first administered on day 2, and the anti-PD-1 antibody is first administered on day 6; the chemotherapy is first administered on day 1, the anti-CD40 antibody is first administered on day 2, and the anti-PD-1 antibody is first administered on day 7; the chemotherapy is first administered on day 1, the anti-CD40 antibody is first administered on day 2, and the anti-PD-1 antibody is first administered on day 8; the chemotherapy is first administered on day 1, the anti-CD40 antibody is first administered on day 2, and the anti-PD-1 antibody is first administered on day 9; the chemotherapy is first administered on day 1, the anti-CD40 antibody is first administered on day 2, and the anti-PD-1 antibody is first administered on day 10; the chemotherapy is first administered on day 1, the anti-CD40 antibody is first administered on day 2, and the anti-PD-1 antibody is first administered on day 11; the chemotherapy is first administered on day 1, the anti-CD40 antibody is first administered on day 2, and the anti-PD-1 antibody is first administered on day 12; the chemotherapy is first administered on day 1, the anti-CD40 antibody is first administered on day 2, and the anti-PD-1 antibody is first administered on day 13; the chemotherapy is first administered on day 1, the anti-CD40 antibody is first administered on day 2, and the anti-PD-1 antibody is first administered on day 14; the chemotherapy is first administered on day 1, the anti-CD40 antibody is first administered on day 2, and the anti-PD-1 antibody is first administered on day 15; the chemotherapy is first administered on day 1, the anti-CD40 antibody is first administered on day 3, and the anti-PD-1 antibody is first administered on day 4; the chemotherapy is first administered on day 1, the anti-CD40 antibody is first administered on day 3, and the anti-PD-1 antibody is first administered on day 5; the chemotherapy is first administered on day 1, the anti-CD40 antibody is first administered on day 3, and the anti-PD-1 antibody is first administered on day 6; the chemotherapy is first administered on day 1, the anti-CD40 antibody is first administered on day 3, and the anti-PD-1 antibody is first administered on day 7; the chemotherapy is first administered on day 1, the anti-CD40 antibody is first administered on day 3, and the anti-PD-1 antibody is first administered on day 8; the chemotherapy is first administered on day 1, the anti-CD40 antibody is first administered on day 3, and the anti-PD-1 antibody is first administered on day 9; the chemotherapy is first administered on day 1, the anti-CD40 antibody is first administered on day 3, and the anti-PD-1 antibody is first administered on day 10; the chemotherapy is first administered on day 1, the anti-CD40 antibody is first administered on day 3, and the anti-PD-1 antibody is first administered on day 11; the chemotherapy is first administered on day 1, the anti-CD40 antibody is first administered on day 3, and the anti-PD-1 antibody is first administered on day 12; the chemotherapy is first administered on day 1, the anti-CD40 antibody is first administered on day 3, and the anti-PD-1 antibody is first administered on day 13; the chemotherapy is first administered on day 1, the anti-CD40 antibody is first administered on day 3, and the anti-PD-1 antibody is first administered on day 14; the chemotherapy is first administered on day 1, the anti-CD40 antibody is first administered on day 3, and the anti-PD-1 antibody is first administered on day 15; the chemotherapy is first administered on day 1, the anti-CD40 antibody is first administered on day 4, and the anti-PD-1 antibody is first administered on day 5; the chemotherapy is first administered on day 1, the anti-CD40 antibody is first administered on day 4, and the anti-PD-1 antibody is first administered on day 6; the chemotherapy is first administered on day 1, the anti-CD40 antibody is first administered on day 4, and the anti-PD-1 antibody is first administered on day 7; the chemotherapy is first administered on day 1, the anti-CD40 antibody is first administered on day 4, and the anti-PD-1 antibody is first administered on day 8; the chemotherapy is first administered on day 1, the anti-CD40 antibody is first administered on day 4, and the anti-PD-1 antibody is first administered on day 9; the chemotherapy is first administered on day 1, the anti-CD40 antibody is first administered on day 4, and the anti-PD-1 antibody is first administered on day 10; the chemotherapy is first administered on day 1, the anti-CD40 antibody is first administered on day 4, and the anti-PD-1 antibody is first administered on day 11; the chemotherapy is first administered on day 1, the anti-CD40 antibody is first administered on day 4, and the anti-PD-1 antibody is first administered on day 12; the chemotherapy is first administered on day 1, the anti-CD40 antibody is first administered on day 4, and the anti-PD-1 antibody is first administered on day 13; the chemotherapy is first administered on day 1, the anti-CD40 antibody is first administered on day 4, and the anti-PD-1 antibody is first administered on day 14; the chemotherapy is first administered on day 1, the anti-CD40 antibody is first administered on day 4, and the anti-PD-1 antibody is first administered on day 15; the chemotherapy is first administered on day 1, the anti-CD40 antibody is first administered on day 5, and the anti-PD-1 antibody is first administered on day 6; the chemotherapy is first administered on day 1, the anti-CD40 antibody is first administered on day 5, and the anti-PD-1 antibody is first administered on day 7; the chemotherapy is first administered on day 1, the anti-CD40 antibody is first administered on day 5, and the anti-PD-1 antibody is first administered on day 8; the chemotherapy is first administered on day 1, the anti-CD40 antibody is first administered on day 5, and the anti-PD-1 antibody is first administered on day 9; the chemotherapy is first administered on day 1, the anti-CD40 antibody is first administered on day 5, and the anti-PD-1 antibody is first administered on day 10; the chemotherapy is first administered on day 1, the anti-CD40 antibody is first administered on day 5, and the anti-PD-1 antibody is first administered on day 11; the chemotherapy is first administered on day 1, the anti-CD40 antibody is first administered on day 5, and the anti-PD-1 antibody is first administered on day 12; the chemotherapy is first administered on day 1, the anti-CD40 antibody is first administered on day 5, and the anti-PD-1 antibody is first administered on day 13; the chemotherapy is first administered on day 1, the anti-CD40 antibody is first administered on day 5, and the anti-PD-1 antibody is first administered on day 14; the chemotherapy is first administered on day 1, the anti-CD40 antibody is first administered on day 5, and the anti-PD-1 antibody is first administered on day 15; the chemotherapy is first administered on day 1, the anti-CD40 antibody is first administered on day 6, and the anti-PD-1 antibody is first administered on day 7; the chemotherapy is first administered on day 1, the anti-CD40 antibody is first administered on day 6, and the anti-PD-1 antibody is first administered on day 8; the chemotherapy is first administered on day 1, the anti-CD40 antibody is first administered on day 6, and the anti-PD-1 antibody is first administered on day 9; the chemotherapy is first administered on day 1, the anti-CD40 antibody is first administered on day 6, and the anti-PD-1 antibody is first administered on day 10; the chemotherapy is first administered on day 1, the anti-CD40 antibody is first administered on day 6, and the anti-PD-1 antibody is first administered on day 11; the chemotherapy is first administered on day 1, the anti-CD40 antibody is first administered on day 6, and the anti-PD-1 antibody is first administered on day 11; the chemotherapy is first administered on day 1, the anti-CD40 antibody is first administered on day 6, and the anti-PD-1 antibody is first administered on day 13; the chemotherapy is first administered on day 1, the anti-CD40 antibody is first administered on day 6, and the anti-PD-1 antibody is first administered on day 14; the chemotherapy is first administered on day 1, the anti-CD40 antibody is first administered on day 6, and the anti-PD-1 antibody is first administered on day 15; the chemotherapy is first administered on day 1, the anti-CD40 antibody is first administered on day 7, and the anti-PD-1 antibody is first administered on day 8; the chemotherapy is first administered on day 1, the anti-CD40 antibody is first administered on day 7, and the anti-PD-1 antibody is first administered on day 9; the chemotherapy is first administered on day 1, the anti-CD40 antibody is first administered on day 7, and the anti-PD-1 antibody is first administered on day 10; the chemotherapy is first administered on day 1, the anti-CD40 antibody is first administered on day 7, and the anti-PD-1 antibody is first administered on day 11; the chemotherapy is first administered on day 1, the anti-CD40 antibody is first administered on day 7, and the anti-PD-1 antibody is first administered on day 12; the chemotherapy is first administered on day 1, the anti-CD40 antibody is first administered on day 7, and the anti-PD-1 antibody is first administered on day 13; the chemotherapy is first administered on day 1, the anti-CD40 antibody is first administered on day 7, and the anti-PD-1 antibody is first administered on day 14; the chemotherapy is first administered on day 1, the anti-CD40 antibody is first administered on day 7, and the anti-PD-1 antibody is first administered on day 15; the chemotherapy is first administered on day 1, the anti-CD40 antibody is first administered on day 8, and the anti-PD-1 antibody is first administered on day 9; the chemotherapy is first administered on day 1, the anti-CD40 antibody is first administered on day 8, and the anti-PD-1 antibody is first administered on day 10; the chemotherapy is first administered on day 1, the anti-CD40 antibody is first administered on day 8, and the anti-PD-1 antibody is first administered on day 11; the chemotherapy is first administered on day 1, the anti-CD40 antibody is first administered on day 8, and the anti-PD-1 antibody is first administered on day 12; the chemotherapy is first administered on day 1, the anti-CD40 antibody is first administered on day 8, and the anti-PD-1 antibody is first administered on day 13; the chemotherapy is first administered on day 1, the anti-CD40 antibody is first administered on day 8, and the anti-PD-1 antibody is first administered on day 14; and the chemotherapy is first administered on day 1, the anti-CD40 antibody is first administered on day 8, and the anti-PD-1 antibody is first administered on day 15.

In some embodiments, the chemotherapy is first administered on day 1, the anti-CD40 antibody is first administered on day 3, and the anti-PD-1 antibody is first administered on day 8. In some embodiments, the chemotherapy is first administered on day 1, the anti-CD40 antibody is first administered on day 5, and the anti-PD-1 antibody is first administered on day 8. In some embodiments, the chemotherapy is first administered on day 1, the anti-CD40 antibody is first administered on day 7, and the anti-PD-1 antibody is first administered on day 8. In some embodiments, the chemotherapy is first administered on day 1, the anti-CD40 antibody is first administered on day 7, and the anti-PD-1 antibody is first administered on day 15. In some embodiments, the chemotherapy is first administered on day 1, the anti-CD40 antibody is first administered on day 8, and the anti-PD-1 antibody is first administered on day 10, day 11, day 12, or day 15. In some embodiments, the chemotherapy is first administered on day 1, the anti-CD40 antibody is first administered on day 8, and the anti-PD-1 antibody is first administered on day 15.

In some embodiments, the chemotherapy is administered in a cycle of every 4 weeks. In some embodiments, the chemotherapy is administered on day 1, day 5, and day 8 of each cycle. In some embodiments, the anti-CD40 antibody is administered in a cycle of every 4 weeks.

In some embodiments, the anti-CD40 antibody is administered at a dose of about 3 µg/kg, about 10 µg/kg, about 30 µg/kg, about 45 µg/kg, or about 60 µg/kg patient body weight. In some embodiments, the anti-CD40 antibody is administered at a dose of about 10 µg/kg patient body weight. In some embodiments, the anti-CD40 antibody is administered at a dose of about 30 µg/kg patient body weight.

In some embodiments, the cancer treated is melanoma; bladder cancer; lung cancer, e.g., small cell lung cancer and non-small cell lung cancer; ovarian cancer; kidney cancer; pancreatic cancer; breast cancer; cervical cancer; head and neck cancer, prostate cancer; glioblastoma; non-hodgkin lymphoma; chronic lymphocytic leukemia; hepatocellular carcinoma; or multiple myeloma. In some embodiments, the cancer treated is melanoma; breast cancer, metastatic breast cancer; lung cancer, non-small cell lung cancer (NSCLC), or pancreatic cancer. In some embodiments, the cancer treated is pancreatic cancer. In some embodiments, the cancer treated is pancreatic ductal adenocarcinoma (PDAC). In some embodiments, the cancer treated is metastatic pancreatic ductal adenocarcinoma.

Other features and advantages of the invention will be apparent from the following detailed description and from the claims.

### DEFINITIONS

The term “combination therapy” or “combination” refers to a treatment regimen including administering more than one therapeutic agent. A combination therapy can include two, three, four, five six, seven, eight, night, ten or a number of therapeutic agents. Each therapeutic agent can be the same or different kind of molecule including, e.g., a biologic agent, a small molecule, an antibody, a chemotherapeutic agent, etc. Each therapeutic agent can be administered in the same or different cycles. Some or all of the therapeutic agents can be formulated together. Some or all of the therapeutic agents can be administered separately.

A “polypeptide” or “polypeptide chain” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein can also comprise non-peptidic components, such as carbohydrate groups. Carbohydrates and other non-peptidic substituents can be added to a protein by the cell in which the protein is produced, and will vary with the type of cell. Proteins are defined herein in terms of their amino acid backbone structures; substituents such as carbohydrate groups are generally not specified, but can be present nonetheless.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “antibody” is used herein to denote immunoglobulin proteins produced by the body in response to the presence of an antigen and that bind to the antigen, as well as antigen-binding fragments and engineered variants thereof. Hence, the term “antibody” includes, for example, intact monoclonal antibodies comprising full-length immunoglobulin heavy and light chains (e.g., antibodies produced using hybridoma technology) and antigen-binding antibody fragments, such as F(ab′)2 and Fab fragments. Genetically engineered intact antibodies and fragments, such as chimeric antibodies, humanized antibodies, single-chain Fv fragments, single-chain antibodies, diabodies, minibodies, linear antibodies, multivalent or multispecific (e.g., bispecific) hybrid antibodies, and the like are also included. Thus, the term “antibody” is used expansively to include any protein that comprises an antigen-binding site of an antibody and is capable of specifically binding to its antigen.

An “antigen-binding site of an antibody” is that portion of an antibody that is sufficient to bind to its antigen. The minimum such region is typically a variable region or a genetically engineered variant thereof. Single-domain binding sites can be generated from camelid antibodies (see Muyldermans and Lauwereys, J. Mol. Recog. 12:131-140, 1999; Nguyen et al., EMBO J. 19:921-930, 2000) or from VH domains of other species to produce single-domain antibodies (“dAbs”; see Ward et al., Nature 341:544-546, 1989; US Pat. No. 6,248,516 to Winter et al.). In certain variations, an antigen-binding site is a polypeptide region having only 2 complementarity determining regions (CDRs) of a naturally or non-naturally (e.g., mutagenized) occurring heavy chain variable region or light chain variable region, or combination thereof (see, e.g., Pessi et al.,Nature 362:367-369, 1993; Qiu et al., Nature Biotechnol. 25:921-929, 2007). More commonly, an antigen-binding site of an antibody comprises both a heavy chain variable (VH) domain and a light chain variable (VL) domain that bind to a common epitope. Within the context of the present disclosure, an antibody can include one or more components in addition to an antigen-binding site, such as, for example, a second antigen-binding site of an antibody (which can bind to the same or a different epitope or to the same or a different antigen), a peptide linker, an immunoglobulin constant region, an immunoglobulin hinge, an amphipathic helix (see Pack and Pluckthun, Biochem. 31:1579-1584, 1992), a non-peptide linker, an oligonucleotide (see Chaudri et al., FEBS Letters 450:23-26, 1999), a cytostatic or cytotoxic drug, and the like, and can be a monomeric or multimeric protein. Examples of molecules comprising an antigen-binding site of an antibody are known in the art and include, for example, Fv, single-chain Fv (scFv), Fab, Fab′, F(ab′)₂, F(ab)_{c}, diabodies, dAbs, minibodies, nanobodies, Fab-scFv fusions, bispecific (scFv)₄-IgG, and bispecific (scFv)₂-Fab. (See, e.g., Hu et al., Cancer Res. 56:3055-3061, 1996; Atwell et al., Molecular Immunology 33:1301-1312, 1996; Carter and Merchant, Curr. Opin. Biotechnol. 8:449-454, 1997; Zuo et al., Protein Engineering 13:361-367, 2000; and Lu et al., J. Immunol. Methods 267:213-226, 2002.)

The terms “cancer”, “cancerous”, or “malignant” refer to or describe the physiological condition in mammals that is typically characterized by unregulated cell growth. The cancer can be a solid tumor or a blood cancer. The cancer can also be a melanoma, a breast cancer, including metastatic breast cancer, a lung cancer, including a non-small cell lung cancer, pancreatic cancer, lymphoma, colorectal cancer, or renal cancer. In some embodiments, the cancer is a melanoma; a breast cancer, including metastatic breast cancer; a lung cancer, including a non-small cell lung cancer; or pancreatic cancer. The pancreatic cancer can be a pancreatic ductal adenocarcinoma (PDAC). The PDAC can also be metastatic.

As used herein, the term “immunoglobulin” refers to a protein consisting of one or more polypeptides substantially encoded by immunoglobulin gene(s). One form of immunoglobulin constitutes the basic structural unit of native (i.e., natural) antibodies in vertebrates. This form is a tetramer and consists of two identical pairs of immunoglobulin chains, each pair having one light chain and one heavy chain. In each pair, the light and heavy chain variable regions (VL and VH) are together primarily responsible for binding to an antigen, and the constant regions are primarily responsible for the antibody effector functions. Five classes of immunoglobulin protein (IgG, IgA, IgM, IgD, and IgE) have been identified in higher vertebrates. IgG comprises the major class; it normally exists as the second most abundant protein found in plasma. In humans, IgG consists of four subclasses, designated IgG1, IgG2, IgG3, and IgG4. The heavy chain constant regions of the IgG class are identified with the Greek symbol γ. For example, immunoglobulins of the IgG1 subclass contain a γ1 heavy chain constant region. Each immunoglobulin heavy chain possesses a constant region that consists of constant region protein domains (CH1, hinge, CH2, and CH3; IgG3 also contains a CH4 domain) that are essentially invariant for a given subclass in a species. DNA sequences encoding human and non-human immunoglobulin chains are known in the art. (See, e.g., Ellison et al., DNA 1:11-18, 1981; Ellison et al., Nucleic Acids Res. 10:4071-4079, 1982; Kenten et al., Proc. Natl. Acad. Sci. USA 79:6661-6665, 1982; Seno et al., Nuc. Acids Res. 11:719-726, 1983; Riechmann et al., Nature 332:323-327, 1988; Amster et al., Nuc. Acids Res. 8:2055-2065, 1980; Rusconi and Kohler, Nature 314:330-334, 1985; Boss et al., Nuc. Acids Res. 12:3791-3806, 1984; Bothwell et al., Nature 298:380-382, 1982; van der Loo et al., Immunogenetics 42:333-341, 1995; Karlin et al., J. Mol. Evol. 22:195-208, 1985; Kindsvogel et al., DNA 1:335-343, 1982; Breiner et al., Gene 18:165-174, 1982; Kondo et al., Eur. J. Immunol. 23:245-249, 1993; and GenBank Accession No. J00228.) For a review of immunoglobulin structure and function, see Putnam, The Plasma Proteins, Vol V, Academic Press, Inc., 49-140, 1987; and Padlan, Mol. Immunol. 31:169-217, 1994. The term “immunoglobulin” is used herein for its common meaning, denoting an intact antibody, its component chains, or fragments of chains, depending on the context.

Full-length immunoglobulin “light chains” (about 25 Kd or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 Kd or 446 amino acids) are encoded by a variable region gene (encoding about 116 amino acids) and a gamma, mu, alpha, delta, or epsilon constant region gene (encoding about 330 amino acids), the latter defining the antibody’s isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin light or heavy chain variable region (also referred to herein as a “light chain variable region” (“VL region”) or “heavy chain variable region” (“VH region”), respectively) consists of a “framework” region interrupted by three hypervariable regions, also called “complementarity determining regions” or “CDRs.” The framework regions serve to align the CDRs for specific binding to an epitope of an antigen. Thus, the term “hypervariable region” or “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4. The assignment of amino acids to each domain is in accordance with the definitions of Kabat, Sequences of Proteins of Immunological Interest (National Institutes of Health, Bethesda, MD, 1987 and 1991), or Chothia & Lesk, J. Mol. Biol. 196:901-917, 1987; Chothia et al., Nature 342:878-883, 1989. Kabat also provides a widely used numbering convention (Kabat numbering) in which corresponding residues between different heavy chains or between different light chains are assigned the same number. CDRs 1, 2, and 3 of a VL domain are also referred to herein, respectively, as CDR-L1, CDR-L2, and CDR-L3; CDRs 1, 2, and 3 of a VH domain are also referred to herein, respectively, as CDR-H1, CDR-H2, and CDR-H3.

Unless the context dictates otherwise, the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

The term “chimeric antibody” refers to an antibody having variable regions derived from a first species and constant regions derived from a second species. Chimeric immunoglobulins or antibodies can be constructed, for example by genetic engineering, from immunoglobulin gene segments belonging to different species. The term “humanized antibody,” as defined infra, is not intended to encompass chimeric antibodies. Although humanized antibodies are chimeric in their construction (i.e., comprise regions from more than one species of protein), they include additional features (i.e., variable regions comprising donor CDR residues and acceptor framework residues) not found in chimeric immunoglobulins or antibodies, as defined herein.

The term “humanized VH domain” or “humanized VL domain” refers to an immunoglobulin VH or VL domain comprising some or all CDRs entirely or substantially from a non-human donor immunoglobulin (e.g., a mouse or rat) and variable region framework sequences entirely or substantially from human immunoglobulin sequences. The non-human immunoglobulin providing the CDRs is called the “donor” and the human immunoglobulin providing the framework is called the “acceptor.” In some instances, humanized antibodies can retain non-human residues within the human variable framework regions to enhance proper binding characteristics (e.g., mutations in the frameworks can be required to preserve binding affinity when an antibody is humanized).

A “humanized antibody” is an antibody comprising one or both of a humanized VH domain and a humanized VL domain. Immunoglobulin constant region(s) need not be present, but if they are, they are entirely or substantially from human immunoglobulin constant regions.

Specific binding of an antibody to its target antigen means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not, however, necessarily imply that a monoclonal antibody binds one and only one target.

With regard to proteins as described herein, reference to amino acid residues corresponding to those specified by SEQ ID NO includes post-translational modifications of such residues.

The term “diluent” as used herein refers to a solution suitable for altering or achieving an exemplary or appropriate concentration or concentrations as described herein.

The term “container” refers to something into which an object or liquid can be placed or contained, e.g., for storage (for example, a holder, receptacle, vessel, or the like).

The term “administration route” includes art-recognized administration routes for delivering a therapeutic protein such as, for example, parenterally, intravenously, intramuscularly, or subcutaneously. For administration of an antibody for the treatment of cancer, administration into the systemic circulation by intravenous or subcutaneous administration can be desired. For treatment of a cancer characterized by a solid tumor, administration can also be localized directly into the tumor, if so desired.

The term “treatment” refers to the administration of a therapeutic agent to a patient, who has a disease with the purpose to cure, heal, alleviate, delay, relieve, alter, remedy, ameliorate, improve or affect the disease.

The term “paclitaxel” refers to the chemotherapeutic agent paclitaxel in original form or various formulations such as “albumin-bound paclitaxel” and “ABRAXANE®” which is a brand name of paclitaxel containing albumin-bound paclitaxel.

The term “patient” includes human and other mammalian subjects that receive either prophylactic or therapeutic treatment.

The term “effective amount,” “effective dose,” or “effective dosage” refers to an amount that is sufficient to achieve or at least partially achieve the desired effect, e.g., sufficient to inhibit the occurrence or ameliorate one or more symptoms of a disease or disorder. An effective amount of a pharmaceutical composition is administered in an “effective regime.” The term “effective regime” refers to a combination of amount of the composition being administered and dosage frequency adequate to accomplish prophylactic or therapeutic treatment of the disease or disorder.

As used herein, the term “about” denotes an approximate range of plus or minus 10% from a specified value. For instance, the language “about 20 µg/kg” encompasses a range of 18-22 µg/kg. As used herein, about also includes the exact amount. Hence “about 20 µg/kg” means “about 20 µg/kg” and also “20 µg/kg.”

As used herein, a “pembrolizumab variant” means a monoclonal antibody which comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g, the variant positions are located in the FR (framework) regions of the variable regions or located in the constant regions, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

“PD-1 antagonist” means any chemical compound or biological molecule that blocks binding of PD-L1 (e.g., expressed on a cancer cell) to PD-1 (e.g., expressed on an immune cell (T cell, B cell or NKT cell)) and preferably also blocks binding of PD-L2 (e.g., expressed on a cancer cell) to PD-1 (e.g., immune-cell expressed PD-1). Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

As used herein, a “SEA-CD40 variant” means a monoclonal antibody which comprises heavy chain and light chain sequences that are substantially identical to those in SEA-CD40, except for having three, two or one conservative amino acid substitutions at positions that are located outside of the light chain CDRs and six, five, four, three, two or one conservative amino acid substitutions that are located outside of the heavy chain CDRs, e.g, the variant positions are located in the FR (framework) regions of the variable regions or located in the constant regions, and optionally has a deletion of the C-terminal lysine residue of the heavy chain. In other words, SEA-CD40 and a SEA-CD40 variant comprise identical CDR sequences, but differ from each other due to having a conservative amino acid substitution at no more than three or six other positions in their full length light and heavy chain sequences, respectively. A SEA-CD40 variant is substantially the same as SEA-CD40 with respect to the following properties: binding affinity to CD40 and non-fucosylation characteristics.

“Conservatively amino acid substitutions” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in the art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1.

TABLE 1

Exemplary Conservative Amino Acid Substitutions

| | |
|---|---|
| Original residue | Conservative substitution |
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1A shows the median tumor volume of mice of a CT26 colon cancer model that were untreated (G1; control); administered with an anti-mPD-1 surrogate antibody (G2; “anti-PD1”); administered with SEA-m1C10 (G3; “SEA-m1C10”); or administered with SEA-m1C10 and the anti-mPD1 surrogate antibody by simultaneous dosing (G4; “SEA-m1C10 + anti-PD1”).

FIG. 1B shows the median tumor volume of mice of a CT26 colon cancer model that were untreated (G1; control); administered with an anti-mPD-1 surrogate antibody (G2; “anti-PD1”); administered with SEA-m1C10 (G3; “SEA-m1C10”); or administered with SEA-m1C10 and the anti-mPD1 surrogate antibody by staggered dosing (G4; “SEA-m1C10 + anti-PD1”). The time period for SEA-m1C10 dosing and anti-PD1 dosing are labeled by vertical lines on the X-axis.

FIG. 2A shows the survival curves of mice of a A20 disseminated lymphoma model that were untreated (G1; control); administered with an anti-mPD-1 surrogate antibody (G2; “anti-PD1”); administered with SEA-m1C10 (G3; “SEA-m1C10”); or administered with SEAm1C10 and the anti-mPD1 surrogate antibody by simultaneous dosing (G4; “SEA-m1C10 + anti-PD1”).

FIG. 2B shows the mean tumor volume of mice of a A20 disseminated lymphoma model that were untreated (G1; control); administered with an anti-mPD-1 surrogate antibody (G2; “anti-PD1”); administered with SEA-m1C10 (G3; “SEA-m1C10”); or administered with SEA-m1C10 and the anti-mPD1 surrogate antibody by staggered dosing (G4; “SEA-m1C10 + anti-PD1”). The time period for SEA-m1C10 dosing and anti-PD1 dosing are labeled by vertical lines on the X-axis.

FIG. 3A shows the mean tumor volume of mice of a RENCA renal cell carcinoma model that were untreated (G1; control); administered with an anti-mPD-1 surrogate antibody (G2; “anti-PD1”); administered with SEA-m1C10 (G3; “SEA-m1C10”); or administered with SEA-m1C10 and the anti-mPD1 surrogate antibody by simultaneous dosing (G4; “SEA-m1C10 + anti-PD1”).

FIG. 3B shows the mean tumor volume of mice of a RENCA renal cell carcinoma model that were untreated (G1; control); administered with an anti-mPD-1 surrogate antibody (G2; “anti-PD1”); administered with SEA-m1C10 (G3; “SEA-m1C10”); or administered with SEA-m1C10 and the anti-mPD1 surrogate antibody by staggered dosing (G4; “SEA-m1C10 + anti-PD1”). The time period for SEA-m1C10 dosing and anti-PD1 dosing are labeled by vertical lines on the X-axis.

FIG. 4 lists sequences discussed in the disclosure. Variable regions are in bold and underlined.

### DETAILED DESCRIPTION

This disclosure relates to methods of treating cancer using a combination of an anti-CD40 antibody such as SEA-CD40, and an anti-PD-1 antibody such as pembrolizumab. In one aspect, the disclosure also provides methods of treating cancer using a combination of an anti-CD40 antibody, an anti-PD-1 antibody, and a chemotherapy.

### Cd40

CD40 is a member of the tumor necrosis factor (TNF) receptor superfamily. It is a single chain type I transmembrane protein with an apparent MW of 50 kDa. Its mature polypeptide core consists of 237 amino acids, of which 173 amino acids comprise an extracellular domain (ECD) organized into 4 cysteine-rich repeats that are characteristic of TNF receptor family members. Two potential N-linked glycosylation sites are present in the membrane proximal region of the ECD, while potential O-linked glycosylation sites are absent. A 22 amino acid transmembrane domain connects the ECD with the 42 amino acid cytoplasmic tail of CD40. Sequence motifs involved in CD40-mediated signal transduction have been identified in the CD40 cytoplasmic tail. These motifs interact with cytoplasmic factors called TNF-R-associated factors (TRAFs) to trigger multiple downstream events including activation of MAP kinases and NFκB, which in turn modulate the transcriptional activities of a variety of inflammation-, survival-, and growth-related genes. See, e.g., van Kooten and Banchereau, J. Leukoc. Biol. 67:2-17 (2000); Elgueta et al., Immunol. Rev. 229:152-172 (2009).

Within the hematopoietic system, CD40 can be found on B cells at multiple stages of differentiation, monocytes, macrophages, platelets, follicular dendritic cells, dendritic cells (DC), eosinophils, and activated T cells. In normal non-hematopoietic tissues, CD40 has been detected on renal epithelial cells, keratinocytes, fibroblasts of synovial membrane and dermal origins, and activated endothelium. A soluble version of CD40 is released from CD40-expressing cells, possibly through differential splicing of the primary transcript or limited proteolysis by the metalloproteinase TNFα converting enzyme. Shed CD40 can potentially modify immune responses by interfering with the CD40/CD40L interaction. See, e.g., van Kooten and Banchereau, J. Leukoc. Biol. 67:2-17 (2000); Elgueta et al., Immunol. Rev. 229:152-172 (2009).

The endogenous ligand for CD40 (CD40L) is a type II membrane glycoprotein of 39 kDa also known as CD154. CD40L is a member of the TNF superfamily and is expressed as a trimer on the cell surface. CD40L is transiently expressed on activated CD4+, CD8+, and γδ T cells. CD40L is also detected at variable levels on purified monocytes, activated B cells, epithelial and vascular endothelial cells, smooth muscle cells, and DCs, but the functional relevance of CD40L expression on these cell types has not been clearly defined (van Kooten 2000; Elgueta 2009). However, expression of CD40L on activated platelets has been implicated in the pathogenesis of thrombotic diseases. See, e.g., Ferroni et al., Curr. Med. Chem. 14:2170-2180 (2007).

The best-characterized function of the CD40/CD40L interaction is its role in contact-dependent reciprocal interaction between antigen-presenting cells and T cells. See, e.g., van Kooten and Banchereau, J. Leukoc. Biol. 67:2-17 (2000); Elgueta et al., Immunol. Rev. 229:152-172 (2009). Binding of CD40L on activated T cells to CD40 on antigen-activated B cells not only drives rapid B cell expansion, but also provides an essential signal for B cells to differentiate into either memory B cells or plasma cells. CD40 signaling is responsible for the formation of germinal centers in which B cells undergo affinity maturation and isotype switching to acquire the ability to produce high affinity antibodies of the IgG, IgA, and IgE isotypes. See, e.g., Kehry, J. Immunol. 156:2345-2348 (1996). Thus, individuals with mutations in the CD40L locus that prevent functional CD40/CD40L interaction suffer from the primary immunodeficiency X-linked hyper-IgM syndrome that is characterized by over-representation of circulating IgM and the inability to produce IgG, IgA, and IgE. These patients demonstrate suppressed secondary humoral immune responses, increased susceptibility to recurrent pyrogenic infections, and a higher frequency of carcinomas and lymphomas. Gene knockout experiments in mice to inactivate either CD40 or CD40L locus reproduce the major defects seen in X-linked hyper-IgM patients. These KO mice also show impaired antigen-specific T cell priming, suggesting that the CD40L/CD40 interaction is also a critical factor for mounting cell-mediated immune responses. See, e.g., Elgueta et al., Immunol. Rev. 229:152-172 (2009).

The immune-stimulatory effects of CD40 ligation by CD40L or anti-CD40 in vivo have correlated with immune responses against syngeneic tumors. See, e.g., French et al., Nat. Med. 5:548-553 (1999). A deficient immune response against tumor cells can result from a combination of factors such as expression of immune checkpoint molecules, such as PD1 or CTLA-4, decreased expression of MHC antigens, poor expression of tumor-associated antigens, appropriate adhesion, or co-stimulatory molecules, and the production of immunosuppressive proteins like TGFβ by the tumor cells. CD40 ligation on antigen presenting and transformed cells results in up-regulation of adhesion proteins (e.g., CD54), co-stimulatory molecules (e.g., CD86) and MHC antigens, as well as inflammatory cytokine secretion, thereby potentially inducing and/or enhancing the antitumor immune response, as well as the immunogenicity of the tumor cells. See, e.g., Gajewski et al., Nat. Immunol. 14:1014-1022 (2013).

A primary consequence of CD40 cross-linking is DC activation (often termed licensing) and potentiation of myeloid and B cells ability to process and present tumor-associated antigens to T cells. Besides having a direct ability to activate the innate immune response, a unique consequence of CD40 signaling is APC presentation of tumor-derived antigens to CD8+ cytotoxic T cell (CTL) precursors in a process known as ‘cross-priming’. This CD40-dependent activation and differentiation of CTL precursors by mature DCs into tumor-specific effector CTLs can enhance cell-mediated immune responses against tumor cells. See, e.g., Kurts et al., Nat. Rev. Immunol. 10:403-414 (2010).

Agonistic CD40 mAbs including dacetuzumab, the SEA-CD40 parent molecule (a fucosylated anti-CD40 antibody), have shown encouraging clinical activity in single-agent and combination chemotherapy settings. Dacetuzumab demonstrated some clinical activity in a phase 1 study in NHL and a phase 2 study in diffuse large B-cell lymphoma (DLBCL). See, e.g., Advani et al., J. Clin. Oncol. 27:4371-4377 (2009) and De Vos et al., J. Hematol. Oncol. 7:1-9 (2014). Additionally CP-870,893, a humanized IgG2 agonist antibody to CD40, showed encouraging activity in solid tumor indications when combined with paclitaxel or carboplatin or gemcitabine. In these studies, activation of antigen presenting cells, cytokine production, and generation of antigen-specific T cells were seen. See, e.g., Beatty et al., Clin. Cancer Res. 19:6286-6295 (2013) and Vonderheide et al., Oncoimmunology 2:e23033 (2013)

### Anti-CD40 Antibodies

Anti-CD40 antibodies, e.g., S2C6, have been disclosed in US20170333556A1, which is herein incorporated by reference. The S2C6 antibody is a partial agonist of the CD40 signaling pathway and thus has the following activities: binding to human CD40 protein, binding to cynomolgus CD40 protein, activation of the CD40 signaling pathway, potentiation of the interaction of CD40 with its ligand, CD40L. See, e.g., U.S. Pat. No. 6,946,129, which is herein incorporated by reference.

Humanized anti-CD40 antibodies, e.g., humanized S2C6 (hS2C6), have been disclosed in US8303955B2 and US8492531B2, both of which are herein incorporated by reference.

Non-fucosylated anti-CD40 antibodies, e.g., hS2C6 or SEA-CD40 have been disclosed in US20170333556A1. In addition to enhanced binding to Fc receptors, SEA-CD40 also enhances activity of the CD40 pathway, as compared to the parent antibody, dacetuzumab. The SEA-CD40 antibody thus, is administered to patients at lower doses and using different schedules of administration.

SEA-CD40 exhibits enhanced binding to FcγIII receptors, and enhanced ability to activate the CD40 signaling pathway in immune cells, as described in US20170333556A1. Methods of making the non-fucosylated antibodies including SEA-CD40 are also disclosed in US20170333556A1.

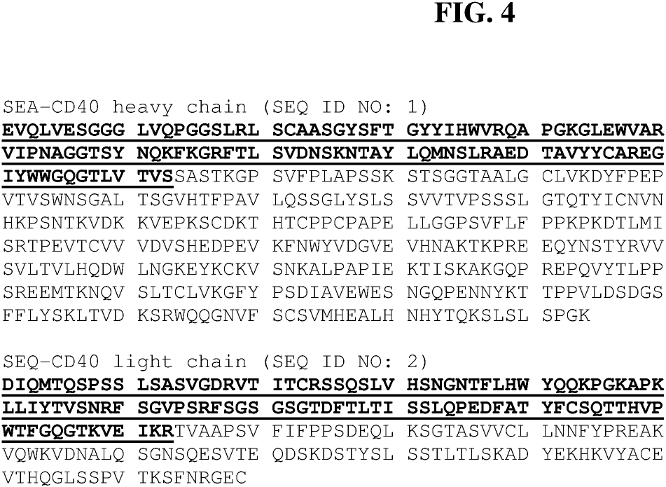

The amino acid sequences of the heavy chain and light chain for SEA-CD40 are disclosed as SEQ ID NOs: 1 and 2, respectively (See FIG. 4). As disclosed in US20170333556A1, the variable region of the heavy chain is from amino acids 1-113 of SEQ ID NO: 1. The variable region of the light chain is from amino acids 1-113 of SEQ ID NO: 2.

In some embodiment, a humanized anti-CD40 antibody disclosed herein is useful in the treatment of various disorders associated with the expression of CD40 as described herein. Because SEA-CD40 activates the immune system to respond against tumor-related antigens, its use is not limited to cancers that express CD40. Thus SEA-CD40 can be used to treat both CD40 positive and CD40 negative cancers.

### Methods of Making Non-Fucosylated Antibodies

This disclosure provides compositions and methods for preparing humanized S2C6 antibodies with reduced core fucosylation. As used herein, “core fucosylation” refers to addition of fucose (“fucosylation”) to N-acetylglucosamine (“GlcNAc”) at the reducing terminal of an N-linked glycan.

Fucosylation of complex N-glycoside-linked sugar chains bound to the Fc region (or domain) of the SEA-CD40 antibody backbone is reduced. As used herein, a “complex N-glycoside-linked sugar chain” is typically bound to asparagine 297 (according to the EU index as set forth in Kabat, “Sequences of Immunological Interest, 5^{th} Ed., Pub. No. 91-3242, U.S. Dept. Healtth & Human Services, NIH, Bethesda, MD, 1991). As used herein, the complex N-glycoside-linked sugar chain has a biantennary composite sugar chain, mainly having the following structure:

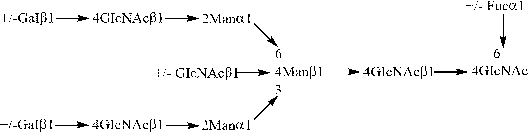

where ± indicates the sugar molecule can be present or absent, and the numbers indicate the position of linkages between the sugar molecules. In the above structure, the sugar chain terminal which binds to asparagine is called a reducing terminal (at right), and the opposite side is called a non-reducing terminal. Fucose is usually bound to N-acetylglucosamine (“GlcNAc”) of the reducing terminal, typically by an α1,6 bond (the 6-position of GlcNAc is linked to the 1-position of fucose). “Gal” refers to galactose, and “Man” refers to mannose.

A “complex N-glycoside-linked sugar chain” includes 1) a complex type, in which the non-reducing terminal side of the core structure has one or more branches of galactose-N-acetylglucosamine (also referred to as “gal-GlcNAc”) and the non-reducing terminal side of Gal-GlcNAc optionally has a sialic acid, bisecting N-acetylglucosamine or the like; or 2) a hybrid type, in which the non-reducing terminal side of the core structure has both branches of a high mannose N-glycoside-linked sugar chain and complex N-glycoside-linked sugar chain.

In some embodiments, the “complex N-glycoside-linked sugar chain” includes a complex type in which the non-reducing terminal side of the core structure has zero, one or more branches of galactose-N-acetylglucosamine (also referred to as “gal-GlcNAc”) and the non-reducing terminal side of Gal-GlcNAc optionally further has a structure such as a sialic acid, bisecting N-acetylglucosamine or the like.

According to the present methods, typically only a minor amount of fucose is incorporated into the complex N-glycoside-linked sugar chain(s) of the SEA-CD40 molecule. For example, in various embodiments, less than about 60%, less than about 50%, less than about 40%, less than about 30%, less than about 20%, less than about 15%, less than about 10%, less than about 5%, or less than about 3% of the antibody has core fucosylation by fucose. In some embodiments, about 2% of the antibody has core fucosylation by fucose.

In certain embodiments, only a minor amount of a fucose analog (or a metabolite or product of the fucose analog) is incorporated into the complex N-glycoside-linked sugar chain(s). For example, in various embodiments, less than about 40%, less than about 30%, less than about 20%, less than about 15%, less than about 10%, less than about 5%, or less than about 3% of the SEA-CD40 antibody has core fucosylation by a fucose analog or a metabolite or product of the fucose analog. In some embodiments, about 2% of the SEA-CD40 antibody has core fucosylation by a fucose analog or a metabolite or product of the fucose analog.

Methods of making non-fucosylated antibodies by incubating antibody-producing cells with a fucose analogue are described, e.g., in WO/2009/135181. Briefly, cells that have been engineered to express the humanized S2C6 antibody are incubated in the presence of a fucose analogue or an intracellular metabolite or product of the fucose analog. As used herein, an intracellular metabolite can be, for example, a GDP-modified analog or a fully or partially de-esterified analog. A product can be, for example, a fully or partially de-esterified analog. In some embodiments, a fucose analogue can inhibit an enzyme(s) in the fucose salvage pathway. For example, a fucose analog (or an intracellular metabolite or product of the fucose analog) can inhibit the activity of fucokinase, or GDP-fucose-pyrophosphorylase. In some embodiments, a fucose analog (or an intracellular metabolite or product of the fucose analog) inhibits fucosyltransferase (preferably a 1,6-fucosyltransferase, e.g., the FUT8 protein). In some embodiments, a fucose analog (or an intracellular metabolite or product of the fucose analog) can inhibit the activity of an enzyme in the de novo synthetic pathway for fucose. For example, a fucose analog (or an intracellular metabolite or product of the fucose analog) can inhibit the activity of GDP-mannose 4,6-dehydratase or/or GDP-fucose synthetase. In some embodiments, the fucose analog (or an intracellular metabolite or product of the fucose analog) can inhibit a fucose transporter (e.g., GDP-fucose transporter).

In some embodiments, the fucose analogue is 2-flurofucose. Methods of using fucose analogues in growth medium and other fucose analogues are disclosed, e.g., in WO/2009/135181, which is herein incorporated by reference.

Other methods for engineering cell lines to reduce core fucosylation included gene knock-outs, gene knock-ins and RNA interference (RNAi). In gene knock-outs, the gene encoding FUT8 (alpha 1,6- fucosyltransferase enzyme) is inactivated. FUT8 catalyzes the transfer of a fucosyl residue from GDP-fucose to position 6 of Asn-linked (N-linked) GlcNac of an N-glycan. FUT8 is reported to be the only enzyme responsible for adding fucose to the N-linked biantennary carbohydrate at Asn297. Gene knock-ins add genes encoding enzymes such as GNTIII or a golgi alpha mannosidase II. An increase in the levels of such enzymes in cells diverts monoclonal antibodies from the fucosylation pathway (leading to decreased core fucosylation), and having increased amount of bisecting N-acetylglucosamines. RNAi typically also targets FUT8 gene expression, leading to decreased mRNA transcript levels or knocking out gene expression entirely. Any of these methods can be used to generate a cell line that can produce a non-fucosylated antibody, e.g., an SEA-CD40 antibody.

Those of skill will recognize that many methods are available to determine the amount of fucosylation on an antibody. Methods include, e.g., LC-MS via PLRP-S chromatography and electrospray ionization quadrupole TOF MS.

The non-fucosylated antibody, SEA-CD40, when adminstered to a patient induces activation of monocyte maturation into macrophages and induce production of cytokines, including, e.g., interferon-γ (IFN- γ) and chemokine that elicit robust T-cell response to immune system challenges. Unlike fully agoninstic antibodies, such as antibody 24.4.1., SEA-CD40 does not induce production of immune-dampening cytokines, such as interleukin-10 (IL-10). IL-10, in turn, induces activity of T-regulatory cells, which dampen the immune resopnse. Thus, SEA-CD40 is useful for induction of a robust T-cell mediated immune response without promoting activity of T-regulatory cells.

In some embodiments, the disclosure relates to a composition comprising a non-fucosylated anti-CD40 antibody such as SEA-CD40, wherein the constant region of the antibody such as SEA-CD40 has an N-glycoside-linked sugar chain at residue N297 according to the EU index; and wherein less than 40%, less than 30%, less than 20%, less than 15%, less than 10%, less than 5%, less than 3%, less than 2% of N-glycoside-linked sugar chains in the composition comprise a fucose residue. In some embodiments, less than 20% of N-glycoside-linked sugar chains in the composition comprise a fucose residue. In some embodiments, less than 10% of N-glycoside-linked sugar chains in the composition comprise a fucose residue. In some embodiments, less than 5% of N-glycoside-linked sugar chains in the composition comprise a fucose residue. In some embodiments, less than 3% of N-glycoside-linked sugar chains in the composition comprise a fucose residue. In some embodiments, less than 2% of N-glycoside-linked sugar chains in the composition comprise a fucose residue.

In some embodiments, the disclosure relates to treating a cancer by administering a composition comprising a non-fucosylated anti-CD40 antibody such as SEA-CD40, wherein the constant region of the antibody such as SEA-CD40 has an N-glycoside-linked sugar chain at residue N297 according to the EU index; and wherein less than 40%, less than 30%, less than 20%, less than 15%, less than 10%, less than 5%, less than 3%, less than 2% of N-glycoside-linked sugar chains in the composition comprise a fucose residue. In some embodiments, less than 20% of N-glycoside-linked sugar chains in the composition comprise a fucose residue. In some embodiments, less than 10% of N-glycoside-linked sugar chains in the composition comprise a fucose residue. In some embodiments, less than 5% of N-glycoside-linked sugar chains in the composition comprise a fucose residue. In some embodiments, less than 3% of N-glycoside-linked sugar chains in the composition comprise a fucose residue. In some embodiments, less than 2% of N-glycoside-linked sugar chains in the composition comprise a fucose residue.

The humanized anti-CD40 antibody or agent is administered by any suitable means, including parenteral, subcutaneous, intraperitoneal, intrapulmonary, and intranasal, and, if desired for local immunosuppressive treatment, intralesional administration (including perfusing or otherwise contacting the graft with the antibody before transplantation). The humanized anti-CD40 antibody or agent can be administered, for example, as an infusion or as a bolus. Parenteral infusions include intramuscular, intravenous, intraarterial, intraperitoneal, or subcutaneous administration. In addition, the humanized anti-CD40 antibody is suitably administered by pulse infusion, particularly with declining doses of the antibody. In one aspect, the dosing is given by injections, most preferably intravenous or subcutaneous injections, depending in part on whether the administration is brief or chronic.

For the prevention or treatment of disease, the appropriate dosage of antibody will depend on a variety of factors such as the type of disease to be treated, as defined above, the severity and course of the disease, whether the antibody is administered for preventive or therapeutic purposes, previous therapy, the patient’s clinical history and response to the antibody, and the discretion of the attending physician. The antibody is suitably administered to the patient at one time or over a series of treatments.

The antibody composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody to be administered will be governed by such considerations, and is the minimum amount necessary to prevent, ameliorate, or treat cancers including cancers. Because SEA-CD40 activates the immune system to respond against tumor-related antigens, its use is not limited to cancers that express CD40. Thus SEA-CD40 can be used to treat both CD40 positive and CD40 negative cancers.

The antibody need not be, but is optionally, formulated with one or more agents currently used to prevent or treat the disorder in question. The effective amount of such other agents depends on the amount of humanized anti-CD40 antibody present in the formulation, the type of disorder or treatment, and other factors discussed above. These are generally used in the same dosages and with administration routes as used hereinbefore or about from 1 to 99% of the heretofore employed dosages.

PD-1 antagonists useful in the treatment method, medicaments and uses of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

Examples of mAbs that bind to human PD-1, and useful in the treatment method, medicaments and uses of the present invention, are described in US7488802, US7521051, US8008449, US8354509, US8168757, WO2004/004771, WO2004/072286, WO2004/056875, and US2011/0271358. Specific anti-human PD-1 mAbs useful as the PD-1 antagonist in the treatment method, medicaments and uses of the present invention include: pembrolizumab (also known as MK-3475), a humanized IgG4 mAb with the structure described in WHO Drug Information, Vol. 27, No. 2, pages 161-162 (2013) and which comprises the heavy and light chain amino acid sequences shown in Table 2; nivolumab (BMS-936558), a human IgG4 mAb with the structure described in WHO Drug Information, Vol. 27, No. 1, pages 68-69 (2013); the humanized antibodies h409A11, h409A16 and h409A17, which are described in WO2008/156712, and AMP-514, which is being developed by MedImmune.

Examples of mAbs that bind to human PD-L1, and useful in the treatment method, medicaments and uses of the present invention, are described in WO2013/019906, WO2010/077634 A1 andUS8383796. Specific anti-human PD-L1 mAbs useful as the PD-1 antagonist in the treatment method, medicaments and uses of the present invention include MPDL3280A, BMS-936559, MEDI4736, MSB0010718C and an antibody which comprises the heavy chain and light chain variable regions of SEQ ID NO: 24 and SEQ ID NO: 21, respectively, of WO2013/019906.

Other PD-1 antagonists useful in the treatment method, medicaments and uses of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

In some preferred embodiments of the treatment method, medicaments and uses of the present invention, the PD-1 antagonist is a monoclonal antibody, or antigen binding fragment thereof, which comprises: (a) light chain CDRs SEQ ID NOs: 3, 4 and 5 and (b) heavy chain CDRs SEQ ID NOs: 6, 7 and 8.

In other preferred embodiments of the treatment method, medicaments and uses of the present invention, the PD-1 antagonist is a monoclonal antibody, or antigen binding fragment thereof, which specifically binds to human PD-1 and comprises (a) a heavy chain variable region comprising SEQ ID NO: 11 or a variant thereof, and (b) a light chain variable region comprising SEQ ID NO: 6 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In another preferred embodiment of the treatment method, medicaments and uses of the present invention, the PD-1 antagonist is a monoclonal antibody which specifically binds to human PD-1 and comprises (a) a heavy chain comprising SEQ ID NO: 12 and (b) a light chain comprising SEQ ID NO: 7.

In all of the above treatment method, medicaments and uses, the PD-1 antagonist inhibits the binding of PD-L1 to PD-1, and preferably also inhibits the binding of PD-L2 to PD-1. In some embodiments of the above treatment method, medicaments and uses, the PD-1 antagonist is a monoclonal antibody, or an antigen binding fragment thereof, which specifically binds to PD-1 or to PD-L1 and blocks the binding of PD-L1 to PD-1. In one embodiment, the PD-1 antagonist is an anti-PD-1 antibody which comprises a heavy chain and a light chain, and wherein the heavy and light chains comprise the amino acid sequences in SEQ ID NO: 12 and SEQ ID NO: 7, respectively.

In one embodiment, the PD-1 antagonist is an anti-PD-1 antibody. In one embodiment, the anti-PD-1 antibody is pembrolizumab. In one embodiment, the anti-PD-1 antibody is a pembrolizumab variant.

Table 2 below provides a list of the amino acid sequences of exemplary anti-PD-1 mAbs for use in the treatment method, medicaments and uses of the present invention.

TABLE 2

Exemplary PD-1 Antibody Sequences

| Antibody Feature | Amino Acid Sequence | SEQ ID NO. |
|---|---|---|
| Pembrolizumab Light Chain | | |
| CDR1 | RASKGVSTSGYSYLH | 3 |
| CDR2 | LASYLES | 4 |
| CDR3 | QHSRDLPLT | 5 |
| Variable Region | EIVLTQSPATLSLSPGERATLSCRASKGVSTSGYSYLHWY QQKPGQAPRLLIYLASYLESGVPARFSGSGSGTDFTLTISS LEPEDFAVYYCQHSRDLPLTFGGGTKVEIK | 6 |
| Light Chain | EIVLTQSPATLSLSPGERATLSCRASKGVSTSGYSYLHWY QQKPGQAPRLLIYLASYLESGVPARFSGSGSGTDFTLTISS LEPEDFAVYYCQHSRDLPLTFGGGTKVEIKRTVAAPSVFI FPPSDEQLKSGTASVVCLLNNFYPREAKVQWKVDNALQ SGNSQESVTEQDSKDSTYSLSSTLTLSKADYEKHKVYAC EVTHQGLSSPVTKSFNRGEC | 7 |
| Pembrolizumab Heavy Chain | | |
| CDR1 | NYYMY | 8 |
| CDR2 | GINPSNGGTNFNEKFKN | 9 |
| CDR3 | RDYRFDMGFDY | 10 |
| Variable Region | QVQLVQSGVEVKKPGASVKVSCKASGYTFTNYYMYWV RQAPGQGLEWMGGINPSNGGTNFNEKFKNRVTLTTDSST TTAYMELKSLQFDDTAVYYCARRDYRFDMGFDYWGQG TTVTVSS | 11 |
| Heavy Chain | QVQLVQSGVEVKKPGASVKVSCKASGYTFTNYYMYWV RQAPGQGLEWMGGINPSNGGTNFNEKFKNRVTLTTDSST TTAYMELKSLQFDDTAVYYCARRDYRFDMGFDYWGQG TTVTVSSASTKGPSVFPLAPCSRSTSESTAALGCLVKDYF PEPVTVSWNSGALTSGVHTFPAVLQSSGLYSLSSVVTVPS SSLGTKTYTCNVDHKPSNTKVDKRVESKYGPPCPPCPAP EFLGGPSVFLFPPKPKDTLMISRTPEVTCVVVDVSQEDPE VQFNWYVDGVEVHNAKTKPREEQFNSTYRVVSVLTVLH QDWLNGKEYKCKVSNKGLPSSIEKTISKAKGQPREPQVY TLPPSQEEMTKNQVSLTCLVKGFYPSDIAVEWESNGQPE NNYKTTPPVLDSDGSFFLYSRLTVDKSRWQEGNVFSCSV MHEALHNHYTQKSLSLSLGK | 12 |

### Dosage and Administration of an anti-CD40 Antibody Such as SEA-CD40 for Treating Cancer

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively antibodies can be in lyophilized form for constitution with a suitable vehicle, e.g., sterile pyrogen-free water, before use.

In some embodiments, an anti-CD40 antibody such as SEA-CD40 is administered intravenously. In some embodiments, an anti-CD40 antibody such as SEA-CD40 is administered subcutaneously. In a further embodiment, an anti-CD40 antibody such as SEA-CD40 is administered subcutaneously at the site of a tumor.

SEA-CD40 is an agonistic antibody and has enhanced binding to Fcγ receptors III and, exhibits enhanced activation of the CD40 signaling pathway. Because of its enhanced activation of the CD40 pathway SEA-CD40 is a potent activator of the immune system. The enhanced activation of the immune system allows SEA-CD40 to be dosed at low levels, as compared to a fucosylated parent antibody.

As an example, an anti-CD40 antibody such as SEA-CD40 can be administered to patients at levels between about 0.1 to about 70 µg/kg (µg antibody per kilogram patient body weight). Other possible dosage ranges include about 1 µg/kg to about 60 µg/kg, about 10 µg/kg to about 50 µg/kg, and about 20 µg/kg to about 40 µg/kg. Other possible dosage ranges include the following: about 1 µg/kg to about 5 µg/kg, about 5 µg/kg to about 10 µg/kg, about 10 µg/kg to about 15 µg/kg, about 15 µg/kg to about 20 µg/kg, about 20 µg/kg to about 25 µg/kg, about 25 µg/kg to about 30 µg/kg, about 30 µg/kg to about 35 µg/kg, about 35 µg/kg to about 40 µg/kg, about 40 µg/kg to about 45 µg/kg, about 45 µg/kg to about 50 µg/kg, about 50 µg/kg to about 55 µg/kg, and about 55 µg/kg to about 60 µg/kg.

In some embodiments, an anti-CD40 antibody such as SEA-CD40 is administered to patients at about 1 µg/kg, about 2 µg/kg, about 3 µg/kg, about 4 µg/kg, about 5 µg/kg, about 6 µg/kg, about 7 µg/kg, about 8 µg/kg, about 9 µg/kg, about 10 µg/kg, about 11 µg/kg, about 12 µg/kg, about 13 µg/kg, about 14 µg/kg, about 15 µg/kg, about 16 µg/kg, about 17 µg/kg, about 18 µg/kg, about 19 µg/kg, about 20 µg/kg, about 21 µg/kg, about 22 µg/kg, about 23 µg/kg, about 24 µg/kg, about 25 µg/kg, about 26 µg/kg, about 27 µg/kg, about 28 µg/kg, about 29 µg/kg, about 30 µg/kg, about 31 µg/kg, about 32 µg/kg, about 33 µg/kg, about 34 µg/kg, about 35 µg/kg, about 36 µg/kg, about 37 µg/kg, about 38 µg/kg, about 39 µg/kg, about 40 µg/kg, about 41 µg/kg, about 42 µg/kg, about 43 µg/kg, about 44 µg/kg, about 45 µg/kg, about 46 µg/kg, about 47 µg/kg, about 48 µg/kg, about 49 µg/kg, about 50 µg/kg, about 51 µg/kg, about 52 µg/kg, about 53 µg/kg, about 54 µg/kg, about 55 µg/kg, about 56 µg/kg, about 57 µg/kg, about 58 µg/kg, about 59 µg/kg, about 60 µg/kg, about 61 µg/kg, about 62 µg/kg, about 63 µg/kg, about 64 µg/kg, about 65 µg/kg, about 66 µg/kg, about 67 µg/kg, about 68 µg/kg, about 69 µg/kg, or about 70 µg/kg. In preferred embodiments, an anti-CD40 antibody such as SEA-CD40 is administered to patients at about 3 µg/kg, about 10 µg/kg, about 30 µg/kg, about 45 µg/kg, or about 60 µg/kg. In a more preferred embodiment, an anti-CD40 antibody such as SEA-CD40 is administered to cancer patients at about 30 µg/kg or about 10 µg/kg. In another more preferred embodiment, an anti-CD40 antibody such as SEA-CD40 is administered to cancer patients at about 10 µg/kg. In yet another more preferred embodiment, an anti-CD40 antibody such as SEA-CD40 is administered to cancer patients at about 30 µg/kg.

An anti-CD40 antibody such as SEA-CD40 can be administered at different intervals including one week intervals, two weeks intervals, three week intervals, four weeks intervals, five week intervals, six week intervals, seven week intervals, eight week intervals, nine weeks, ten weeks, eleven weeks, twelve weeks, etc. In other words, an anti-CD40 antibody such as SEA-CD40 can be administered every week, every two weeks, every three weeks, every four weeks, every five weeks, every six weeks, every seven weeks, every eight weeks, every nine weeks, every ten weeks, every eleven weeks, every twelve weeks etc. In some embodiments, intervals are on a monthly schedule, e.g., one month intervals, two month intervals, or three month intervals. In some embodiments, intervals are based on cycles wherein each cycle can comprise one or more administrations of an anti-CD40 antibody such as SEA-CD40. Exemplary lengths of each cycle include one week, two weeks, three weeks, four weeks, five week, six weeks, seven weeks, eight weeks, nine weeks, ten weeks, eleven weeks, and twelve weeks. The lengths of cycles can be different from one cycle to the next. An anti-CD40 antibody such as SEA-CD40 can be administered on any one or more days in each cycle. In some embodiments, an anti-CD40 antibody such as SEA-CD40 is administered on the first day of a cycle. In some embodiments, an anti-CD40 antibody such as SEA-CD40 is administered on the first day of a cycle of three weeks in a treatment period of one cycle, two cycles, three cycles, four cycles, five cycles, six cycles, seven cycles, eight cycles, nine cycles, ten cycles, eleven cycles, twelve cycles, thirteen cycles, fourteen cycles, fifteen cycles, or sixteen cycles.

An anti-CD40 antibody such as SEA-CD40 can be administered on day 1, day 2, day 3, day 4, day 5, day 6, or day 7 of each 1-week cycle, i.e., an anti-CD40 antibody such as SEA-CD40 is administered every week starting on day 1, day 2, day 3, day 4, day 5, day 6, or day 7 of a treatment regimen. An anti-CD40 antibody such as SEA-CD40 can be administered on day 1, day 2, day 3, day 4, day 5, day 6, day 7, day 8, day 9, day 10, day 11, day 12, day 13, or day 14 of each 2-week cycle, i.e., an anti-CD40 antibody such as SEA-CD40 is administered every two weeks starting on day 1, day 2, day 3, day 4, day 5, day 6, day 7, day 8, day 9, day 10, day 11, day 12, day 13, or day 14 of a treatment regimen. An anti-CD40 antibody such as SEA-CD40 can be administered on day 1, day 2, day 3, day 4, day 5, day 6, day 7, day 8, day 9, day 10, day 11, day 12, day 13, day 14, day 15, day 16, day 17, day 18, day 19, day 20, or day 21 of each 3-week cycle, i.e., an anti-CD40 antibody such as SEA-CD40 is administered every three weeks starting on day 1, day 2, day 3, day 4, day 5, day 6, day 7, day 8, day 9, day 10, day 11, day 12, day 13, day 14, day 15, day 16, day 17, day 18, day 19, day 20, or day 21 of a treatment regimen. An anti-CD40 antibody such as SEA-CD40 can be administered on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, or day 28 of each 4-week cycle, i.e., an anti-CD40 antibody such as SEA-CD40 is administered every four weeks starting on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, or day 28 of a treatment regimen. An anti-CD40 antibody such as SEA-CD40 can be administered on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 30, day 31, day 32, day 33, day 34, or day 35 of each 5-week cycle, i.e., an anti-CD40 antibody such as SEA-CD40 is administered every five weeks starting on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 30, day 31, day 32, day 33, day 34, or day 35 of a treatment regimen. An anti-CD40 antibody such as SEA-CD40 can be administered on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 30, day 31, day 32, day 33, day 34, day 35, day 36, day 37, day 38, day 39, day 40, day 41, or day 42 of each 6-week cycle, i.e., an anti-CD40 antibody such as SEA-CD40 is administered every six weeks starting on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 30, day 31, day 32, day 33, day 34, day 35, day 36, day 37, day 38, day 39, day 40, day 41, or day 42 of a treatment regimen.

In this disclosure, administration cycle is described in terms of days or weeks interchangeably as a skilled person would understand. For example, an 1-week administration cycle is the same as a 7-day administration cycle; a 2-week administration cycle is the same as a 14-day administration cycle; a 3-week administration cycle is the same as a 21-week administration cycle; etc.

### Dosage and Administration of Pembrolizumab for Treating Cancer in Combination With the Anti-CD40 Antibody

The pembrolizumab can be administered at 200 mg or 2 mg/kg once every three weeks. In some embodiments, the pembrolizumab is administered at 400 mg once every six weeks.

Pembrolizumab can be administered at different intervals including three week intervals and six week intervals. In other words, pembrolizumab can be administered every three weeks or every six weeks. In some embodiments, intervals are based on cycles wherein each cycle can comprise one or more administrations of pembrolizumab. Exemplary lengths of each cycle include three weeks and six weeks. The lengths of cycles can be different from one cycle to the next. Pembrolizumab can be administered on any one or more days in each cycle.

In some embodiments, as an alternative to pembrolizumab, another anti-PD-1 antibody or an anti-PD-L1 antibody is used. In some embodiments, the anti-PD-1 antibody is selected from the group consisting of Nivolumab, Cemiplimab-rwlc, Spartalizumab, AK105, Tislelizumab, Dostarlimab, MEDI0680, Pidilizumab, AMP-224, and SHR-1210. In some embodiments, the anti-PD-1 antibody is Pembrolizumab, Nivolumab, or Cemiplimab-rwlc. In some embodiments, the anti-PDL1 antibody is selected from the group consisting of Atezolizumab, Durvalumab, Avelumab, SHR-1316, MEDI4736, BMS-936559/MDX-1105, MSB0010718C, MPDL3280A, or Envafolimab. In some embodiments, the anti-PDL1 antibody is Atezolizumab, Durvalumab, or Avelumab.

### Anti-CD40 Antibody and Pembrolizumab Combination Therapy for Treating Cancer

An anti-CD40 antibody such as SEA-CD40 can be used in combination with pembrolizumab for treating cancer.

A treating regime comprising administering an anti-CD40 antibody such as SEA-CD40 and administering pembrolizumab can have different dosing schedules. In some embodiments, an anti-CD40 antibody such as SEA-CD40 is administered on a two-week cycle and pembrolizumab is administered on a three-week cycle. In some embodiments, an anti-CD40 antibody such as SEA-CD40 is administered on a three-week cycle and pembrolizumab is also administered on a three-week cycle. In some embodiments, an anti-CD40 antibody such as SEA-CD40 is administered on a four-week cycle and pembrolizumab is administered on a three-week cycle. In some embodiments, an anti-CD40 antibody such as SEA-CD40 is administered on a two-week cycle and pembrolizumab is administered on a six-week cycle. In some embodiments, an anti-CD40 antibody such as SEA-CD40 is administered on a three-week cycle and pembrolizumab is administered on a six-week cycle. In some embodiments, an anti-CD40 antibody such as SEA-CD40 is administered on a four-week cycle and pembrolizumab is administered on a six-week cycle. In a preferred embodiment, an anti-CD40 antibody such as SEA-CD40 is administered on a four-week cycle and pembrolizumab is administered on a three-week cycle. In another preferred embodiment, an anti-CD40 antibody such as SEA-CD40 is administered on a four-week cycle and pembrolizumab is administered on a six-week cycle.

In a preferred embodiment, an anti-CD40 antibody such as SEA-CD40 is administered before the administration of pembrolizumab. Giving pembrolizumab after SEA-CD40 may be beneficial, as this timing mitigates the potential for pembrolizumab to bind to immune cells with subsequent immune depletion arising from enhanced clearance of pembrolizumab-bound cells by SEA-CD40. In a combination therapy, the first day of each drug’s first cycle of administration all start on the same day.

In some embodiments, an anti-CD40 antibody such as SEA-CD40 is administered on day 1 of the first cycle of the anti-CD40 antibody, and pembrolizumab is administered on day 2 of the first cycle of pembrolizumab. In some embodiments, an anti-CD40 antibody such as SEA-CD40 is administered on day 1 of the first cycle of the anti-CD40 antibody, and pembrolizumab is administered on day 3 of the first cycle of pembrolizumab. In some embodiments, an anti-CD40 antibody such as SEA-CD40 is administered on day 1 of the first cycle of the anti-CD40 antibody, and pembrolizumab is administered on day 4 of the first cycle of pembrolizumab. In some embodiments, an anti-CD40 antibody such as SEA-CD40 is administered on day 1 of the first cycle of the anti-CD40 antibody, and pembrolizumab is administered on day 5 of the first cycle of pembrolizumab. In some embodiments, an anti-CD40 antibody such as SEA-CD40 is administered on day 1 of the first cycle of the anti-CD40 antibody, and pembrolizumab is administered on day 6 of the first cycle of pembrolizumab. In some embodiments, an anti-CD40 antibody such as SEA-CD40 is administered on day 1 of the first cycle of the anti-CD40 antibody, and pembrolizumab is administered on day 7 of the first cycle of pembrolizumab. In some embodiments, an anti-CD40 antibody such as SEA-CD40 is administered on day 1 of the first cycle of the anti-CD40 antibody, and pembrolizumab is administered on day 8 of the first cycle of pembrolizumab. In some embodiments, an anti-CD40 antibody such as SEA-CD40 is administered on day 1 of the first cycle of the anti-CD40 antibody, and pembrolizumab is administered on day 9 of the first cycle of pembrolizumab. In some embodiments, an anti-CD40 antibody such as SEA-CD40 is administered on day 1 of the first cycle of the anti-CD40 antibody, and pembrolizumab is administered on day 10 of the first cycle of pembrolizumab. In some embodiments, an anti-CD40 antibody such as SEA-CD40 is administered on day 1 of the first cycle of the anti-CD40 antibody, and pembrolizumab is administered on day 11 of the first cycle of pembrolizumab. In some embodiments, an anti-CD40 antibody such as SEA-CD40 is administered on day 1 of the first cycle of the anti-CD40 antibody, and pembrolizumab is administered on day 12 of the first cycle of pembrolizumab. In some embodiments, an anti-CD40 antibody such as SEA-CD40 is administered on day 1 of the first cycle of the anti-CD40 antibody, and pembrolizumab is administered on day 13 of the first cycle of pembrolizumab. In some embodiments, an anti-CD40 antibody such as SEA-CD40 is administered on day 1 of the first cycle of the anti-CD40 antibody, and pembrolizumab is administered on day 14 of the first cycle of pembrolizumab.

In some embodiments, an anti-CD40 antibody such as SEA-CD40 is administered on day 2 of the first cycle of the anti-CD40 antibody, and pembrolizumab is administered on day 3 of the first cycle of pembrolizumab. In some embodiments, an anti-CD40 antibody such as SEA-CD40 is administered on day 2 of the first cycle of the anti-CD40 antibody, and pembrolizumab is administered on day 4 of the first cycle of pembrolizumab. In some embodiments, an anti-CD40 antibody such as SEA-CD40 is administered on day 2 of the first cycle of the anti-CD40 antibody, and pembrolizumab is administered on day 5 of the first cycle of pembrolizumab. In some embodiments, an anti-CD40 antibody such as SEA-CD40 is administered on day 2 of the first cycle of the anti-CD40 antibody, and pembrolizumab is administered on day 6 of the first cycle of pembrolizumab. In some embodiments, an anti-CD40 antibody such as SEA-CD40 is administered on day 2 of the first cycle of the anti-CD40 antibody, and pembrolizumab is administered on day 7 of the first cycle of pembrolizumab. In some embodiments, an anti-CD40 antibody such as SEA-CD40 is administered on day 2 of the first cycle of the anti-CD40 antibody, and pembrolizumab is administered on day 8 of the first cycle of pembrolizumab. In some embodiments, an anti-CD40 antibody such as SEA-CD40 is administered on day 2 of the first cycle of the anti-CD40 antibody, and pembrolizumab is administered on day 9 of the first cycle of pembrolizumab. In some embodiments, an anti-CD40 antibody such as SEA-CD40 is administered on day 2 of the first cycle of the anti-CD40 antibody, and pembrolizumab is administered on day 10 of the first cycle of pembrolizumab. In some embodiments, an anti-CD40 antibody such as SEA-CD40 is administered on day 2 of the first cycle of the anti-CD40 antibody, and pembrolizumab is administered on day 11 of the first cycle of pembrolizumab. In some embodiments, an anti-CD40 antibody such as SEA-CD40 is administered on day 2 of the first cycle of the anti-CD40 antibody, and pembrolizumab is administered on day 12 of the first cycle of pembrolizumab. In some embodiments, an anti-CD40 antibody such as SEA-CD40 is administered on day 2 of the first cycle of the anti-CD40 antibody, and pembrolizumab is administered on day 13 of the first cycle of pembrolizumab. In some embodiments, an anti-CD40 antibody such as SEA-CD40 is administered on day 2 of the first cycle of the anti-CD40 antibody, and pembrolizumab is administered on day 14 of the first cycle of pembrolizumab.

In some embodiments, an anti-CD40 antibody such as SEA-CD40 is administered on day 3 of the first cycle of the anti-CD40 antibody, and pembrolizumab is administered on day 4 of the first cycle of pembrolizumab. In some embodiments, an anti-CD40 antibody such as SEA-CD40 is administered on day 3 of the first cycle of the anti-CD40 antibody, and pembrolizumab is administered on day 5 of the first cycle of pembrolizumab. In some embodiments, an anti-CD40 antibody such as SEA-CD40 is administered on day 3 of the first cycle of the anti-CD40 antibody, and pembrolizumab is administered on day 6 of the first cycle of pembrolizumab. In some embodiments, an anti-CD40 antibody such as SEA-CD40 is administered on day 3 of the first cycle of the anti-CD40 antibody, and pembrolizumab is administered on day 7 of the first cycle of pembrolizumab. In some embodiments, an anti-CD40 antibody such as SEA-CD40 is administered on day 3 of the first cycle of the anti-CD40 antibody, and pembrolizumab is administered on day 8 of the first cycle of pembrolizumab. In some embodiments, an anti-CD40 antibody such as SEA-CD40 is administered on day 3 of the first cycle of the anti-CD40 antibody, and pembrolizumab is administered on day 9 of the first cycle of pembrolizumab. In some embodiments, an anti-CD40 antibody such as SEA-CD40 is administered on day 3 of the first cycle of the anti-CD40 antibody, and pembrolizumab is administered on day 10 of the first cycle of pembrolizumab. In some embodiments, an anti-CD40 antibody such as SEA-CD40 is administered on day 3 of the first cycle of the anti-CD40 antibody, and pembrolizumab is administered on day 11 of the first cycle of pembrolizumab. In some embodiments, an anti-CD40 antibody such as SEA-CD40 is administered on day 3 of the first cycle of the anti-CD40 antibody, and pembrolizumab is administered on day 12 of the first cycle of pembrolizumab. In some embodiments, an anti-CD40 antibody such as SEA-CD40 is administered on day 3 of the first cycle of the anti-CD40 antibody, and pembrolizumab is administered on day 13 of the first cycle of pembrolizumab. In some embodiments, an anti-CD40 antibody such as SEA-CD40 is administered on day 3 of the first cycle of the anti-CD40 antibody, and pembrolizumab is administered on day 14 of the first cycle of pembrolizumab.

In some embodiments, an anti-CD40 antibody such as SEA-CD40 is administered on day 4 of the first cycle of the anti-CD40 antibody, and pembrolizumab is administered on day 5 of the first cycle of pembrolizumab. In some embodiments, an anti-CD40 antibody such as SEA-CD40 is administered on day 4 of the first cycle of the anti-CD40 antibody, and pembrolizumab is administered on day 6 of the first cycle of pembrolizumab. In some embodiments, an anti-CD40 antibody such as SEA-CD40 is administered on day 4 of the first cycle of the anti-CD40 antibody, and pembrolizumab is administered on day 7 of the first cycle of pembrolizumab. In some embodiments, an anti-CD40 antibody such as SEA-CD40 is administered on day 4 of the first cycle of the anti-CD40 antibody, and pembrolizumab is administered on day 8 of the first cycle of pembrolizumab. In some embodiments, an anti-CD40 antibody such as SEA-CD40 is administered on day 4 of the first cycle of the anti-CD40 antibody, and pembrolizumab is administered on day 9 of the first cycle of pembrolizumab. In some embodiments, an anti-CD40 antibody such as SEA-CD40 is administered on day 4 of the first cycle of the anti-CD40 antibody, and pembrolizumab is administered on day 10 of the first cycle of pembrolizumab. In some embodiments, an anti-CD40 antibody such as SEA-CD40 is administered on day 4 of the first cycle of the anti-CD40 antibody, and pembrolizumab is administered on day 11 of the first cycle of pembrolizumab. In some embodiments, an anti-CD40 antibody such as SEA-CD40 is administered on day 4 of the first cycle of the anti-CD40 antibody, and pembrolizumab is administered on day 12 of the first cycle of pembrolizumab. In some embodiments, an anti-CD40 antibody such as SEA-CD40 is administered on day 4 of the first cycle of the anti-CD40 antibody, and pembrolizumab is administered on day 13 of the first cycle of pembrolizumab. In some embodiments, an anti-CD40 antibody such as SEA-CD40 is administered on day 4 of the first cycle of the anti-CD40 antibody, and pembrolizumab is administered on day 14 of the first cycle of pembrolizumab.

In some embodiments, an anti-CD40 antibody such as SEA-CD40 is administered on day 5 of the first cycle of the anti-CD40 antibody, and pembrolizumab is administered on day 6 of the first cycle of pembrolizumab. In some embodiments, an anti-CD40 antibody such as SEA-CD40 is administered on day 5 of the first cycle of the anti-CD40 antibody, and pembrolizumab is administered on day 7 of the first cycle of pembrolizumab. In some embodiments, an anti-CD40 antibody such as SEA-CD40 is administered on day 5 of the first cycle of the anti-CD40 antibody, and pembrolizumab is administered on day 8 of the first cycle of pembrolizumab. In some embodiments, an anti-CD40 antibody such as SEA-CD40 is administered on day 5 of the first cycle of the anti-CD40 antibody, and pembrolizumab is administered on day 9 of the first cycle of pembrolizumab. In some embodiments, an anti-CD40 antibody such as SEA-CD40 is administered on day 5 of the first cycle of the anti-CD40 antibody, and pembrolizumab is administered on day 10 of the first cycle of pembrolizumab. In some embodiments, an anti-CD40 antibody such as SEA-CD40 is administered on day 5 of the first cycle of the anti-CD40 antibody, and pembrolizumab is administered on day 11 of the first cycle of pembrolizumab. In some embodiments, an anti-CD40 antibody such as SEA-CD40 is administered on day 5 of the first cycle of the anti-CD40 antibody, and pembrolizumab is administered on day 12 of the first cycle of pembrolizumab. In some embodiments, an anti-CD40 antibody such as SEA-CD40 is administered on day 5 of the first cycle of the anti-CD40 antibody, and pembrolizumab is administered on day 13 of the first cycle of pembrolizumab. In some embodiments, an anti-CD40 antibody such as SEA-CD40 is administered on day 5 of the first cycle of the anti-CD40 antibody, and pembrolizumab is administered on day 14 of the first cycle of pembrolizumab.

In some embodiments, an anti-CD40 antibody such as SEA-CD40 is administered on day 6 of the first cycle of the anti-CD40 antibody, and pembrolizumab is administered on day 7 of the first cycle of pembrolizumab. In some embodiments, an anti-CD40 antibody such as SEA-CD40 is administered on day 6 of the first cycle of the anti-CD40 antibody, and pembrolizumab is administered on day 8 of the first cycle of pembrolizumab. In some embodiments, an anti-CD40 antibody such as SEA-CD40 is administered on day 6 of the first cycle of the anti-CD40 antibody, and pembrolizumab is administered on day 9 of the first cycle of pembrolizumab. In some embodiments, an anti-CD40 antibody such as SEA-CD40 is administered on day 6 of the first cycle of the anti-CD40 antibody, and pembrolizumab is administered on day 10 of the first cycle of pembrolizumab. In some embodiments, an anti-CD40 antibody such as SEA-CD40 is administered on day 6 of the first cycle of the anti-CD40 antibody, and pembrolizumab is administered on day 11 of the first cycle of pembrolizumab. In some embodiments, an anti-CD40 antibody such as SEA-CD40 is administered on day 6 of the first cycle of the anti-CD40 antibody, and pembrolizumab is administered on day 12 of the first cycle of pembrolizumab. In some embodiments, an anti-CD40 antibody such as SEA-CD40 is administered on day 6 of the first cycle of the anti-CD40 antibody, and pembrolizumab is administered on day 13 of the first cycle of pembrolizumab. In some embodiments, an anti-CD40 antibody such as SEA-CD40 is administered on day 6 of the first cycle of the anti-CD40 antibody, and pembrolizumab is administered on day 14 of the first cycle of pembrolizumab.

In some embodiments, an anti-CD40 antibody such as SEA-CD40 is administered on day 7 of the first cycle of the anti-CD40 antibody, and pembrolizumab is administered on day 8 of the first cycle of pembrolizumab. In some embodiments, an anti-CD40 antibody such as SEA-CD40 is administered on day 7 of the first cycle of the anti-CD40 antibody, and pembrolizumab is administered on day 9 of the first cycle of pembrolizumab. In some embodiments, an anti-CD40 antibody such as SEA-CD40 is administered on day 7 of the first cycle of the anti-CD40 antibody, and pembrolizumab is administered on day 10 of the first cycle of pembrolizumab. In some embodiments, an anti-CD40 antibody such as SEA-CD40 is administered on day 7 of the first cycle of the anti-CD40 antibody, and pembrolizumab is administered on day 11 of the first cycle of pembrolizumab. In some embodiments, an anti-CD40 antibody such as SEA-CD40 is administered on day 7 of the first cycle of the anti-CD40 antibody, and pembrolizumab is administered on day 12 of the first cycle of pembrolizumab. In some embodiments, an anti-CD40 antibody such as SEA-CD40 is administered on day 7 of the first cycle of the anti-CD40 antibody, and pembrolizumab is administered on day 13 of the first cycle of pembrolizumab. In some embodiments, an anti-CD40 antibody such as SEA-CD40 is administered on day 7 of the first cycle of the anti-CD40 antibody, and pembrolizumab is administered on day 14 of the first cycle of pembrolizumab.

In some embodiments, an anti-CD40 antibody such as SEA-CD40 is administered on day 8 of the first cycle of the anti-CD40 antibody, and pembrolizumab is administered on day 9 of the first cycle of pembrolizumab. In some embodiments, an anti-CD40 antibody such as SEA-CD40 is administered on day 8 of the first cycle of the anti-CD40 antibody, and pembrolizumab is administered on day 10 of the first cycle of pembrolizumab. In some embodiments, an anti-CD40 antibody such as SEA-CD40 is administered on day 8 of the first cycle of the anti-CD40 antibody, and pembrolizumab is administered on day 11 of the first cycle of pembrolizumab. In some embodiments, an anti-CD40 antibody such as SEA-CD40 is administered on day 8 of the first cycle of the anti-CD40 antibody, and pembrolizumab is administered on day 12 of the first cycle of pembrolizumab. In some embodiments, an anti-CD40 antibody such as SEA-CD40 is administered on day 8 of the first cycle of the anti-CD40 antibody, and pembrolizumab is administered on day 13 of the first cycle of pembrolizumab. In some embodiments, an anti-CD40 antibody such as SEA-CD40 is administered on day 8 of the first cycle of the anti-CD40 antibody, and pembrolizumab is administered on day 14 of the first cycle of pembrolizumab.

In some embodiments, an anti-CD40 antibody such as SEA-CD40 is administered on day 9 of the first cycle of the anti-CD40 antibody, and pembrolizumab is administered on day 10 of the first cycle of pembrolizumab. In some embodiments, an anti-CD40 antibody such as SEA-CD40 is administered on day 9 of the first cycle of the anti-CD40 antibody, and pembrolizumab is administered on day 11 of the first cycle of pembrolizumab. In some embodiments, an anti-CD40 antibody such as SEA-CD40 is administered on day 9 of the first cycle of the anti-CD40 antibody, and pembrolizumab is administered on day 12 of the first cycle of pembrolizumab. In some embodiments, an anti-CD40 antibody such as SEA-CD40 is administered on day 9 of the first cycle of the anti-CD40 antibody, and pembrolizumab is administered on day 13 of the first cycle of pembrolizumab. In some embodiments, an anti-CD40 antibody such as SEA-CD40 is administered on day 9 of the first cycle of the anti-CD40 antibody, and pembrolizumab is administered on day 14 of the first cycle of pembrolizumab.

In some embodiments, an anti-CD40 antibody such as SEA-CD40 is administered on day 10 of the first cycle of the anti-CD40 antibody, and pembrolizumab is administered on day 11 of the first cycle of pembrolizumab. In some embodiments, an anti-CD40 antibody such as SEA-CD40 is administered on day 10 of the first cycle of the anti-CD40 antibody, and pembrolizumab is administered on day 12 of the first cycle of pembrolizumab. In some embodiments, an anti-CD40 antibody such as SEA-CD40 is administered on day 10 of the first cycle of the anti-CD40 antibody, and pembrolizumab is administered on day 13 of the first cycle of pembrolizumab. In some embodiments, an anti-CD40 antibody such as SEA-CD40 is administered on day 10 of the first cycle of the anti-CD40 antibody, and pembrolizumab is administered on day 14 of the first cycle of pembrolizumab.

In some embodiments, an anti-CD40 antibody such as SEA-CD40 is administered on day 11 of the first cycle of the anti-CD40 antibody, and pembrolizumab is administered on day 12 of the first cycle of pembrolizumab. In some embodiments, an anti-CD40 antibody such as SEA-CD40 is administered on day 11 of the first cycle of the anti-CD40 antibody, and pembrolizumab is administered on day 13 of the first cycle of pembrolizumab. In some embodiments, an anti-CD40 antibody such as SEA-CD40 is administered on day 11 of the first cycle of the anti-CD40 antibody, and pembrolizumab is administered on day 14 of the first cycle of pembrolizumab.

In some embodiments, an anti-CD40 antibody such as SEA-CD40 is administered on day 12 of the first cycle of the anti-CD40 antibody, and pembrolizumab is administered on day 13 of the first cycle of pembrolizumab. In some embodiments, an anti-CD40 antibody such as SEA-CD40 is administered on day 12 of the first cycle of the anti-CD40 antibody, and pembrolizumab is administered on day 14 of the first cycle of pembrolizumab.

In some embodiments, an anti-CD40 antibody such as SEA-CD40 is administered on day 13 of the first cycle of the anti-CD40 antibody, and pembrolizumab is administered on day 14 of the first cycle of pembrolizumab.

In some embodiments, the first administration of an anti-CD40 antibody such as SEA-CD40 in the first cycle is 1 day, 2 days, 3 days, 4 days, 5 days, 6 days, 7 days, 8 days, 9 days, 10 days, 11 days, 12 days, 13 days, or 14 days prior to the first administration of pembrolizumab in the first cycle. In some embodiments, the first administration of pembrolizumab is 1 day, days, 3 days, 4 days, 5 days, 6 days, 7 days, 8 days, 9 days, 10 days, 11 days, 12 days, 13 days, or 14 days after the first administration of an anti-CD40 antibody such as SEA-CD40 in the first cycle.

In some embodiments, each dose of the anti-PD-1 antibody is administered at least 1, 2, 3, 4, 5, 6, 7, 8, or 9 days after a dose of the anti-CD40 antibody. In some embodiments, the anti-PD-1 antibody and the anti-CD40 antibody are not administered on the same day. In some embodiments, the interval between the administration of the anti-PD-1 antibody and the administration of the anti-CD40 antibody is at least or about 1, 2, 3, 4, 5, 6, 7, 8, 9, 10, 12, or 24 hours, or at least or about 1, 2, 3, 4, 5, 6, 7, 8, 9, 10, 11, 12, 13, or 14 days.

In some embodiments, pembrolizumab is administered in a cycle of about every 2-4 weeks (e.g., about every 2 weeks, about every 3 weeks, or about every 4 weeks). In some embodiments, pembrolizumab is administered in a cycle of every 14 days, every 15 days, every 16 days, every 17 days, every 18 days, every 19 days, every 20 days, every 21 days, every 22 days, every 23 days, every 24 days, every 25 days, every 26 days, every 27 days, or every 28 days. In some embodiments, pembrolizumab is administered in a cycle of about every 5-7 weeks (e.g., about every 5 weeks, about every 6 weeks, or about every 7 weeks). In some embodiments, pembrolizumab is administered in a cycle of every 35 days, every 36 days, every 37 days, every 38 days, every 39 days, every 40 days, every 41 days, every 42 days, every 43 days, every 44 days, every 45 days, every 46 days, every 47 days, every 48 days, or every 49 days.

In some embodiments, pembrolizumab is administered every 3 weeks at a dose of about 200 mg. In some embodiments, pembrolizumab is administered every 3 weeks at a dose of 200 mg. In some embodiments, pembrolizumab is administered every3 at a dose of about 2 mg/kg. In some embodiments, pembrolizumab is administered every 6 weeks at a dose of 400 mg.

### Combination Therapy With anti-CD40 Antibody and Chemotherapy

The combination therapy of an anti-CD40 antibody such as SEA-CD40 can be combined with chemotherapy. In some embodiments, the combination therapy of an anti-CD40 antibody such as SEA-CD40 and pembrolizumab can be further combined with chemotherapy.

In most humans, millions of cells die via apoptosis and are removed without generating an immune response. However, after treatment with some chemotherapeutic agents, immune cells have been observed to infiltrate tumors. Thus, some tumor cells killed by chemotherapeutic agents act as vaccines and raise a tumor-specific immune response. This phenomenon is referred to as immunogenic cell death (ICD). See, e.g., Kroemer et al., Annu. Rev. Immunol., 31:51-72 (2013). The ability of a chemotherapeutic agent to induce ICD can be determined experimentally. Two criteria must be met. First, injection of an immunocompetent mouse with cancer cells that have been treated in vitro with a chemotherapeutic agent must elicit a protective immune response that is specific for tumor antigens, in the absence of adjuvant. Second, ICD occurring in vivo, e.g., a mouse syngeneic model with treatment using a potential ICD-inducing chemotherapeutic agent, must drive an immune response in the tumor that is dependent on the immune system.

Chemotherapeutic agents that induce ICD include, e.g., anthracyclines, anti-EGFR antibodies, bortezomib, cyclophosphamide, gemcitabine, irradiation of the tumor, and oxaliplatin. A combination of an anti-CD40 antibody such as SEA-CD40 and pembrolizumab can be used in combination with any of these chemotherapy agents to generate an enhanced immune response and treat cancer in a patient. In some embodiments, the combination of an anti-CD40 antibody such as SEA-CD40 and pembrolizumab is used in combination with one or more of gemcitabine, dacarbazine, temozolomide, paclitaxel, albumin-bound paclitaxel (nab-paclitaxel), or carboplatin. ABRAXANE® is a brand name of paclitaxel containing albumin-bound paclitaxel. In some embodiments, the combination of an anti-CD40 antibody such as SEA-CD40 and pembrolizumab is used in combination with both the chemotherapeutic agent gemcitabine and paclitaxel/nab-paclitaxel.

In some embodiments, the combination therapy includes an anti-CD40 antibody such as SEA-CD40, pembrolizumab, and chemotherapy. In some embodiments, chemotherapy used in the combination includes gemcitabine or paclitaxel. In some embodiments, chemotherapy used in the combination includes both gemcitabine and paclitaxel. In some embodiments, paclitaxel is nab-paclitaxel, e.g., ABRAXANE®.

Chemotherapy used in the combination can be administered in cycles. In some embodiments, the cycle is 1 week, i.e., chemotherapy is administered every week. In some embodiments, the cycle is 2 weeks, i.e., chemotherapy is administered every 2 weeks. In some embodiments, the cycle is 3 weeks, i.e., chemotherapy is administered every 3 weeks. In some embodiments, the cycle is 4 weeks, i.e., chemotherapy is administered every 4 weeks. In some embodiments, the cycle is 5 weeks, i.e., chemotherapy is administered every 5 weeks. In some embodiments, the cycle is 6 weeks, i.e., chemotherapy is administered every 6 weeks. In some embodiments, the cycle is 7 weeks, i.e., chemotherapy is administered every 7 weeks. In some embodiments, the cycle is 8 weeks, i.e., chemotherapy is administered every 8 weeks. In each cycle, chemotherapy can be administered one or more times.

In some embodiments, chemotherapy used in the combination is administered in a 4 week cycle, i.e., chemotherapy is administered every 4 weeks, wherein chemotherapy is administered three times in each cycle. In some embodiments, chemotherapy administered in the combination is administered on days 1, 8, and 15 in each cycle.

In some embodiments, chemotherapy used in the combination is administered in a cycle of about every 3-5 weeks (e.g., about every 3 weeks, about every 4 weeks, or about every 5 weeks). In some embodiments, chemotherapy used in the combination is administered in a cycle of every 21 days, every 22 days, every 23 days, every 24 days, every 25 days, every 26 days, every 27 days, every 28 days, every 29 days, every 30 days, every 31 days, every 32 days, every 33 days, every 34 days, or every 35 days.

In some embodiments, chemotherapy used in the combination includes gemcitabine (e.g., INFUGEM™) and/or paclitaxel (e.g., ABRAXANE®). In some embodiments, gemcitabine is administered 1 time, 2 times, 3 times, 4 times, or 5 times in each cycle. In some embodiments, gemcitabine is administered on days 1, 8, and 15 in each cycle (e.g., a 28-day cycle). In some embodiments, gemcitabine is administered on days 1 and 8 of each cycle (e.g., a 21-day cycle). In some embodiments, gemcitabine is administered at a dose of about 800-1500 mg/m² (e.g., about 800 mg/m², about 850 mg/m², about 900 mg/m², about 950 mg/m², about 1000 mg/m², about 1050 mg/m², about 1100 mg/m², about 1150 mg/m², about 1200 mg/m², about 1250 mg/m², about 1300 mg/m², about 1350 mg/m², about 1400 mg/m², about 1450 mg/m², or about 1500 mg/m²), e.g., over about 20-60 minutes (e.g., about 20 minutes, about 30 minutes, about 40 minutes, about 50 minutes, or about 60 minutes). In some embodiments, paclitaxel is administered 1 time, 2 times, 3 times, 4 times, or 5 times in each cycle. In some embodiments, paclitaxel is administered on days 1, 8, and 15 of each cycle (e.g., a 21-day cycle, or a 28-day cycle). In some embodiments, paclitaxel is administered at a dose of about 50-300 mg/m² (e.g., about 50 mg/m², about 60 mg/m², about 70 mg/m², about 80 mg/m², about 90 mg/m², about 100 mg/m², about 110 mg/m², about 120 mg/m², about 125 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg//m², about 250 mg/m², about 260 mg/m², about 270 mg/m², about 280 mg/m², about 290 mg/m², or about 300 mg/m²), e.g., over about 20-60 minutes (e.g., about 20 minutes, about 30 minutes, about 40 minutes, about 50 minutes, or about 60 minutes).

In a preferred embodiment, the first administration of the first cycle of chemotherapy is given prior to the administration of an anti-CD40 antibody such as SEA-CD40 to allow for antigen release to occur from tumor cells as a consequence of chemotherapy. In some embodiments, the chemotherapy is given 1 day prior to the administration of an anti-CD40 antibody such as SEA-CD40. In some embodiments, the chemotherapy is given 2 days prior to the administration of an anti-CD40 antibody such as SEA-CD40. In some embodiments, the chemotherapy is given 3 days prior to the administration of an anti-CD40 antibody such as SEA-CD40. This timing is anticipated to enhance the potential for an anti-CD40 antibody such as SEA-CD40 to lead to an anti-tumor immune response. Specifically, an anti-CD40 antibody such as SEA-CD40 can stimulate antigen update and presentation--and thus is expected to be most effective in the setting of increased levels of circulating antigen. Additionally, waiting 1-3 days after chemotherapy before administering an anti-CD40 antibody such as SEA-CD40 may mitigate the potential for synergistic toxicity.

In some embodiments, each dose of the anti-CD40 antibody is administered at least 1, 2, 3, 4, 5, 6, 7, 8, or 9 days after a dose of the chemotherapy. In some embodiments, the chemotherapy and the anti-CD40 antibody are not administered on the same day. In some embodiments, the interval between the administration of the chemotherapy and the administration of the anti-CD40 antibody is at least or about 1, 2, 3, 4, 5, 6, 7, 8, 9, 10, 12, or 24 hours, or at least or about 1, 2, 3, 4, 5, 6, 7, 8, 9, 10, 11, 12, 13, or 14 days.

In some embodiments, the chemotherapy is administered on day 1 of the first cycle of the chemotherapy, an anti-CD40 antibody such as SEA-CD40 is administered on day 3 of the first cycle of the anti-CD40 antibody, and pembrolizumab is administered on day 8 of the first cycle of pembrolizumab, wherein day 1 of the chemotherapy cycle, the anti-CD40 antibody cycle, and the pembrolizumab cycle starts on the same day.

In a preferred embodiment, the combination therapy includes a chemotherapy administered on day 1, day 8, and day 16 of a 28 day cycle, an anti-CD40 antibody such as SEA-CD40 administered on day 3 of a 28 day cycle, and pembrolizumab administered on day 8 of a 42 day cycle. In some embodiments, the combination therapy includes a chemotherapy administered on day 1, day 8, and day 16 of a 28 day cycle, an anti-CD40 antibody such as SEA-CD40 administered on day 3 of a 28 day cycle, and pembrolizumab administered on day 8 of a 21 day cycle. In a preferred embodiment, the combination therapy includes a chemotherapy administered on day 1, day 8, and day 15 of a 28 day cycle, an anti-CD40 antibody such as SEA-CD40 administered on day 3 of a 28 day cycle, and pembrolizumab administered on day 8 of a 42 day cycle. In some embodiments, the combination therapy includes a chemotherapy administered on day 1, day 8, and day 15 of a 28 day cycle, an anti-CD40 antibody such as SEA-CD40 administered on day 3 of a 28 day cycle, and pembrolizumab administered on day 8 of a 21 day cycle. In some embodiments, the chemotherapy includes both gemcitabine and paclitaxel. In some embodiments, paclitaxel is nab-paclitaxel, e.g., ABRAXANE®.

In one aspect, the disclosure relates to treating a pancreatic cancer with a combination of chemotherapy, pembrolizumab, and SEA-CD40, wherein the chemotherapy is administered on days 1, 8, and 15 of each 28-day cycle, wherein SEA-CD40 is administered on day 3 of each 28-day cycle, and wherein pembrolizumab is administered on day 8 of each 42-day cycle. In some embodiments, the chemotherapy consists of gemcitabine and nab-paclitaxel (ABRAXANE®). In some embodiments, SEA-CD40 is administered intravenously. In some embodiments, SEA-CD40 is administered subcutaneously. In some embodiments, pembrolizumab is administered at 400 mg. In some embodiments, pembrolizumab is administered at 200 mg. In some embodiments, SEA-CD40 is administered at 10 µg/kg. In some embodiments, SEA-CD40 is administered at 30 µg/kg. In some embodiments, the pancreatic cancer is pancreatic ductal adenocarcinoma (PDAC). In some embodiments, the pancreatic cancer is metastatic pancreatic ductal adenocarcinoma (PDAC).

In another aspect, this disclosure relates to treating a pancreatic cancer with a combination of chemotherapy, an anti-PD-1 antibody, and SEA-CD40, wherein the chemotherapy is administered on days 1, 8, and 15 of each each 28-day cycle, wherein SEA-CD40 is administered on day 3 of each 28-day cycle, and wherein the anti-PD-1 antibody is administered on day 8 of each 42-day cycle. In some embodiments, the chemotherapy consists of gemcitabine and nab-paclitaxel (ABRAXANE®). In some embodiments, SEA-CD40 is administered intravenously. In some embodiments, SEA-CD40 is administered subcutaneously. In some embodiments, SEA-CD40 is administered at 10 µg/kg. In some embodiments, SEA-CD40 is administered at 30 µg/kg. In some embodiments, the pancreatic cancer is pancreatic ductal adenocarcinoma (PDAC). In some embodiments, the pancreatic cancer is metastatic pancreatic ductal adenocarcinoma (PDAC). In some embodiments, the anti-PD-1 antibody is pembrolizumab. In some embodiments, pembrolizumab is administered at 400 mg. In some embodiments, pembrolizumab is administered at 200 mg.

### Cancer

A combination therapy of an anti-CD40 antibody such as SEA-CD40, pembrolizumab, and chemotherapy can be used for treating various types of cancer including, e.g., a solid tumor or a blood cancer. In some embodiments, the cancer is melanoma, breast cancer, including metastatic breast cancer, lung cancer, including non-small cell lung cancer, pancreatic cancer, lymphoma; colorectal cancer; or renal cancer. In some embodiments, the cancer is a melanoma; a breast cancer, including metastatic breast cancer; a lung cancer, including a non-small cell lung cancer; or pancreatic cancer. In some embodiments, the pancreatic cancer is a pancreatic ductal adenocarcinoma (PDAC). In some embodiments, the PDAC is metastatic.

Pancreatic cancer has one of the highest mortality rates among all cancers and is the fourth most common cause of adult cancer death in the United States with an estimated 42,470 cases per year. See Nieto et al., The Oncologist, 13:562-576 (2008); and Cancer Facts and Figures, American Cancer Society (2009). It accounts for about 3% of all newly diagnosed cancers in the United States each year. However, almost double that number cancer patients, about 6%, die from pancreatic cancer. See Cancer Facts and Figures, American Cancer Society (2009). The high mortality rate from pancreatic cancer is a result of the high incidence of metastatic disease at the time of diagnosis. As a result, only 5%-15% of patients are candidates present with tumors are amenable to resection. See Nieto et al, The Oncologist, 13:562-576 (2008).

In a preferred embodiment, the combination therapy of an anti-CD40 antibody such as SEA-CD40, pembrolizumab, and chemotherapy can be used for treating pancreatic cancer. In some embodiments, the pancreatic cancer is metastatic pancreatic ductal adenocarcinoma (PDAC).

In some embodiments, the combination therapy of an anti-CD40 antibody such as SEA-CD40, pembrolizumab, and chemotherapy is used to treat tumors that are known to be immune responsive, particularly if the cancer expresses low levels of CD40 or does not detectably express CD40. Immune responsive cancers include, e.g., melanoma; bladder cancer; lung cancer, e.g., small cell lung cancer and non-small cell lung cancer; ovarian cancer; kidney cancer; pancreatic cancer; breast cancer; cervical cancer; head and neck cancer, prostate cancer; glioblastoma; non-hodgkin lymphoma; chronic lymphocytic leukemia; hepatocellular carcinoma; and multiple myeloma.

In some embodiments, the combination therapy of an anti-CD40 antibody such as SEA-CD40, pembrolizumab, and chemotherapy is used to treat solid tumors. In a further embodiment, SEA-CD40 is used to treat blood cancers, e.g., lymphoma, including non-Hodgkin lymphoma and Hodgkin lymphoma; chronic lymphocytic leukemia; or multiple myeloma.

The present disclosure also provides methods of manufacturing the combination therapies for various uses as described herein. The combination therapy can be included in a container, pack, kit, or dispenser together with instructions for administration.

Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications can be practiced within the scope of the appended claims.

### EXAMPLES

The invention is further described in the following examples, which do not limit the scope of the invention described in the claims.

### Example 1: Treating Cancer Patients With a Combination of SEA-CD40, Pembrolizumab, and Chemotherapy

Treatment of pancreatic cancer is being assessed with the combination of chemotherapy, SEA-CD40, and pembrolizumab. Chemotherapy is given on day 1 to stimulate antigen release, followed by SEA-CD40 on day 3. Waiting 1-2 days after chemotherapy before giving SEA-CD40 allows for antigen release to occur from tumor cells as a consequence of chemotherapy. This timing is anticipated to enhance the potential for SEA-CD40 to lead to an anti-tumor immune response. Specifically, SEA-CD40 can stimulate antigen update and presentation--and thus is expected to be most effective in the setting of increased levels of circulating antigen. Additionally, waiting 1-2 days after chemotherapy before administering SEA-CD40 may mitigate the potential for synergistic toxicity. Pembrolizumab is given on day 8. Giving pembrolizumab after SEA-CD40 may be beneficial, as this timing mitigates the potential for pembrolizumab to bind to immune cells with subsequent immune depletion arising from enhanced clearance of pembrolizumab-bound cells by SEA-CD40. SEA-CD40 is dosed at a level that results in significant immune stimulation in humans (e.g. 10 µg/kg or 30 µg/kg).

Background: SEA-CD40 is an investigational non-fucosylated, humanized 1gG1 monoclonal antibody directed against CD40, a co-stimulatory receptor expressed on antigen-presenting cells (APCs). Activation of CD40 on APCs upregulates cytokine production and co-stimulatory receptors, enhancing tumor antigen presentation to T cells. Preclinical data indicate that treatment of pancreatic ductal adenocarcinoma (PDAC) with chemotherapy in conjunction with a CD40 agonist could enhance antigen presentation and initiate an antitumor immune response (Byrne KT and Vonderheide RH, Cell Rep 2016;15, 2719-32). An ongoing Phase 1 study (SGNS40-001) is evaluating SEA-CD40 as monotherapy and in combination with pembrolizumab in patients with advanced solid or hematologic malignancies. A new cohort is enrolling to evaluate the combination of SEA-CD40, gemcitabine, nab-paclitaxel, and pembrolizumab in metastatic PDAC. ABRAXANEⓇ is a brand name of paclitaxel containing albumin-bound paclitaxel.

Methods: The cohort consists of patients with metastatic PDAC who have had no prior therapy for metastatic disease. Patients must be 18 years old or older, with (neo)adjuvant therapy completed > 4 months prior to enrollment, ECOG (Eastern Cooperative Oncology Group) status less than or equals to 1, adequate renal, hepatic, and hematologic function, and measurable disease per RECIST v 1.1 criteria. A standard regimen of gemcitabine and nab-paclitaxel on Days 1, 8, and 15 of each 28-day cycle is administered with SEA-CD40 intravenously (IV) on Day 3. Pembrolizumab is administered every 42 days starting on Day 8. The primary objective is to evaluate the antitumor activity of the administration regimen, and the secondary objectives are to assess the safety and tolerability of SEA-CD40 and pembrolizumab by pharmacokinetic analysis. Efficacy endpoints are confirmed according to RECIST (Response Evaluation Criteria in Solid Tumors) ORR (objective response rate/overall response rate) per investigator (primary), disease control rate (response or stable disease ~16 weeks), duration of response, PFS (progression-free survival), and OS (objective response/overall response). Disease is assessed every 8 weeks using RECIST (Response Evaluation Criteria in Solid Tumors) and immune-based RECIST (iRECIST). Treatment continues until occurrence of unacceptable toxicity, progressive disease per iRECIST, consent withdrawal, or study closure. Assessment of dose-limiting toxicity will occur initially in groups of 6 patients to identify the recommended phase 2 dose of SEA-CD40 for the cohort. Table 3 below illustrates days of administration of chemotherapy, SEA-CD40, and pembrolizumab for the initial 84 days (12 weeks; 3 cycles of 28-day cycle for chemotherapy and SEA-CD40; 2 cycles of 42-day cycle for pembrolizumab). The days of administration of the following days after day 84 follow the same scheme.

TABLE 3

| | | | |
|---|---|---|---|
| | Chemotherapy (gemcitabine and nab-paclitaxel); 28 day-cycle | SEA-CD40; 28 day-cycle | Pembrolizumab; 42 day-cycle |
| Day 1 | 1st cycle 1st administration | | |
| Day 3 | | 1st cycle administration | |
| Day 8 | 1st cycle 2nd administration | | 1st cycle administration |
| Day 15 | 1st cycle 3rd administration | | |
| Day 29 | 2nd cycle 1st administration | | |
| Day 31 | | 2nd cycle administration | |
| Day 36 | 2nd cycle 2nd administration | | |
| Day 43 | 2nd cycle 3rd administration | | |
| Day 50 | | | 2nd cycle administration |
| Day 57 | 3rd cycle 1st administration | | |
| Day 59 | | 3rd cycle administration | |
| Day 64 | 3rd cycle 2nd administration | | |
| Day 71 | 3rd cycle 3rd administration | | |

### Example 2: Murine Tumor Models With Concomitant or Staggered Dosing of a SEA-CD40 Surrogate and/or an Anti-mPD-1 Surrogate Antibody

Mouse models have been proven to be very useful in assessing efficacy and mechanisms of cancer therapeutics. Study of SEA-CD40 in murine cancer models has been difficult because SEA-CD40 does not recognize murine CD40. Therefore, to assess the activity of the non-fucosylated anti-CD40 antibodies, syngeneic murine tumor models were developed. The murine functional equivalents of human IgG1 and human FcγRIII/CD16 are murine IgG2a and murine FcyRIV, respectively, and binding of murine IgG2a to murine FcyRIV mediates antibody-dependent cellular cytotoxicity (ADCC). See, e.g., Bruhns, Blood 119:5640-5649 (2012) and Nimmeriahn et al., Immunity 23:41-51 (2005). The rat antibody 1C10 was used to generate a surrogate of SEA-CD40. See, e.g., Heath et al., Eur. J. Immunol. 24:1828-1834 (1994). Briefly, the VL and VH gene fragments of a rat monoclonal antibody can recognize murine CD40. The 1C10 antibody were cloned in-frame 5′ to murine Ckappa and murine IgG2a CH1-CH2-CH3 fragments, respectively. Expression of the resulting genes in CHO cells generated a chimeric 1C10 antibody with rat VL and VH domains and murine light and heavy chain domains of the IgG2a isotype (mIgG2a 1C10). mIgG2a 1C10 was expressed in the presence of 2-fluorofucose in the CHO cell growth medium using the methods described in U.S. Pat.Application Publication No. US 2017/0333556 A1, to generate a non-fucosylated form of mIgG2a 1C10 (mIgG2a SEA 1C10, or SEA-m1C10).

The single agent activity of SEA-m1C10 or an anti-mPD-1 surrogate antibody (“anti-PD1”), and a combination thereof, were investigated in solid and syngeneic tumor models. Based on SEA-CD40′s mechanism (e.g., enhanced activation of antigen presenting cells, and subsequent induction of an amplified anti-tumor T cell response), SEA-m1C10 was administered prior to the initial treatment with the anti-mPD-1 surrogate antibody.

Stock solutions of the antibodies were diluted to the appropriate concentration and then injected into animals in a volume of 100 µl. Final dosages were 1 mg/kg for SEA-m1C10, and 1 mg/kg for the anti-mPD-1 surrogate antibody. Tumor length, tumor width, and mouse weight were measured throughout the experimental period, and tumor volume was calculated. Euthanasia was performed when tumor volume of a mouse reached 1000 mm³.

### CT26 Colon Cancer Model

The combinatorial activity of the SEA-m1C10 antibody and the anti-mPD-1 surrogate antibody was tested in a CT26 colon cancer model, which is responsive to anti-mPD-1 surrogate antibody treatment. BALB/c mice were implanted with the CT26 syngeneic tumor cell line subcutaneously in the flank of mice on day 0. When the mean tumor size (measured using the formula: Volume (mm³) = 0.5 * Length * Width², wherein length is the longer dimension and width is the shorter dimension) reached 100 mm³, mice were randomly placed into a control group G1 and three treatment groups G2-G4 (5 mice per group).

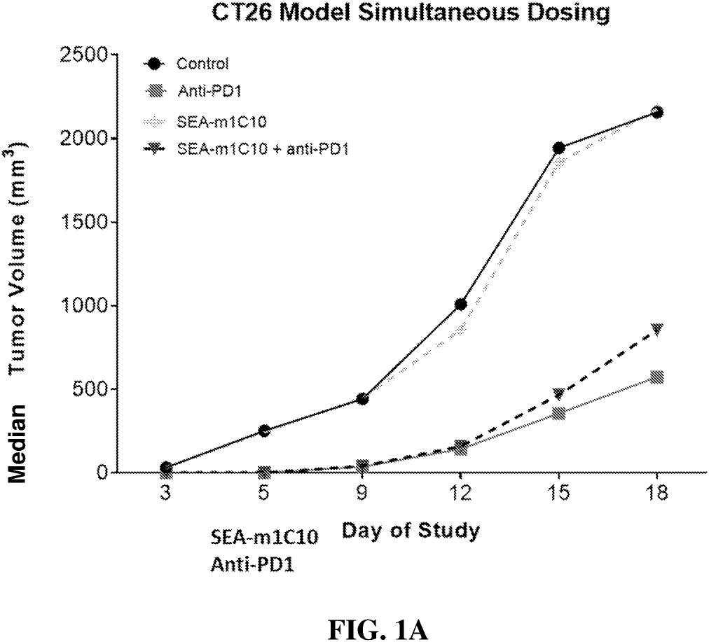

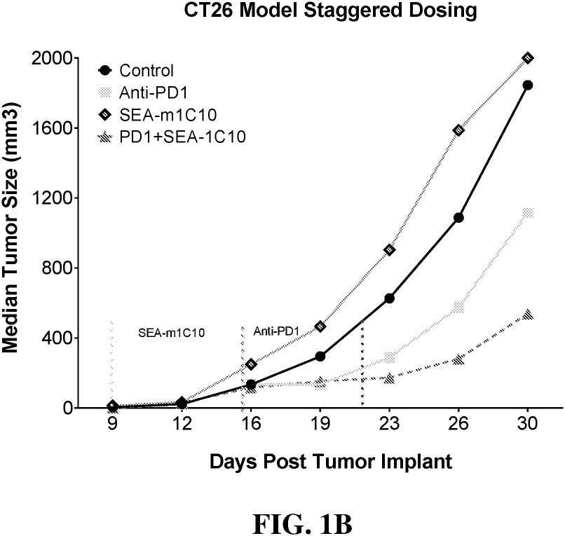

In one experiment, the treatment group mice were administered intraperitoneally with either a single agent (the anti-mPD-1 surrogate antibody (G2) or SEA-m1C10 (G3)), or a combination thereof (G4) on the same day. The administration frequency was once every three days for a total of three treatments. The control group mice (G1) were untreated. Median tumor volume of the mice are shown in FIG. 1A.

Alternatively, mice in treatment groups G3 and G4 were administered with 3 doses of SEA-m1C10 three days apart (e.g., within the period from day 9 to day 15). On the last day of SEA-m1C10 treatment (e.g., on day 15), the first dose of the anti-mPD-1 surrogate antibody was administered to the G2 and G4 group mice, which then received 2 additional doses three days apart (e.g., within the period from day 15 to day 21). The control group mice (G1) were untreated. Median tumor volume of the mice are shown in FIG. 1B.

The results showed that SEA-m1C10 did not exhibit any anti-tumor effect when administered alone. As shown in FIG. 1A, no combinatorial activity, or even an antagonistic activity, was observed when SEA-m1C10 and the anti-mPD-1 surrogate antibody were administered concomitantly. However, the anti-tumor activity was enhanced when SEA-m1C10 was administered in a staggered manner with the anti-mPD-1 surrogate antibody (FIG. 1B). The above results indicate that the timing of administration of these agents is important to achieve a therapeutic benefit. In addition, the results are in line with the proposed mechanism of action of SEA-CD40.

### A20 Disseminated Lymphoma Model

The combinatorial activity of the SEA-m1C10 antibody and the anti-mPD-1 surrogate antibody was also tested in an A20 lymphoma model. BALB/c mice were injected intravenously with A20 cells which established a disseminated lymphoma in about 2-4 weeks. A20 disseminated lymphoma model was initiated in immune-competent female BALB/c mice by injecting 1 × 10⁶ A20 cells per mouse intravenously (IV). Mice were randomly placed into a control group G1 and three treatment groups G2-G4 (6 mice per group).

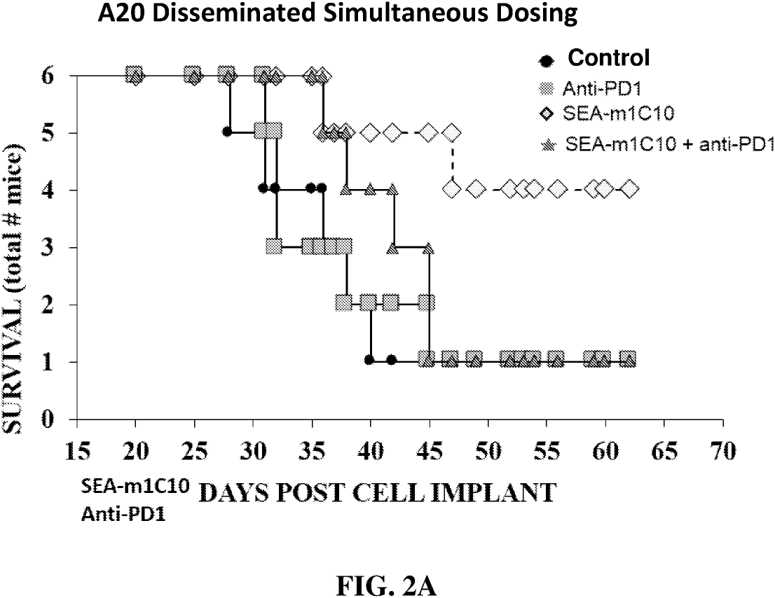

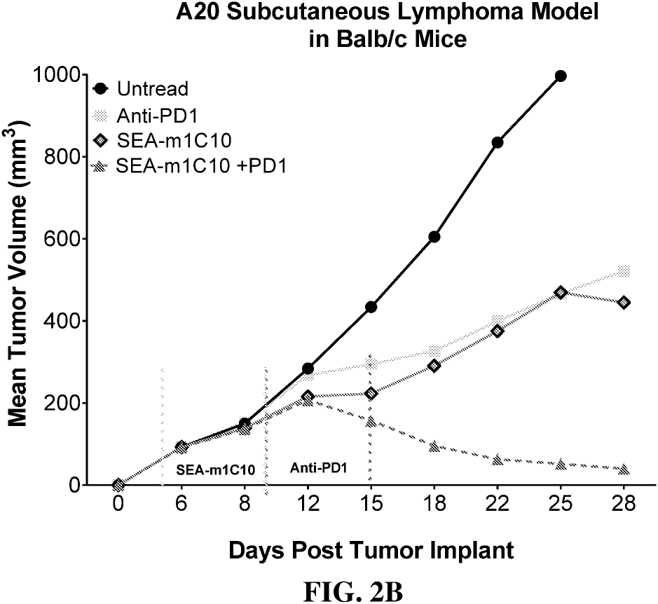

In one experiment, the treatment group mice were administered with antibodies at 3 mg/kg intraperitoneally (i.p.) on a q3dx3 schedule (once every three days for a total of three treatments) starting on day 7 post tumor cell inoculation. The control group mice were untreated. All mice were monitored for weight loss and symptoms of tumor burden, e.g., ascites in the peritoneum. The tumor burden was further verified after sacrificing the mice. FIG. 2A shows the survival curves of the control group mice (G1), mice treated with the anti-mPD-1 surrogate antibody (G2), SEA-m1C10 (G3), and a combination thereof on the same days (G4).

In a different experiment, an A20 lymphoma model was established via subcutaneous methodology. Specifically, tumors were allowed to grow to about 100 mm³ and mice in treatment groups G3 and G4 were administered with 3 doses of SEA-m1C10 three days apart (e.g., within the period from day 4 to day 10). On the last day of SEA-m1C10 treatment, the first dose of the anti-mPD1 surrogate antibody was administered (e.g., on day 10) to the G2 and G4 group mice, which then received 2 additional doses three days apart (e.g., within the period from day 10 to day 16). The control group mice (G1) were untreated. Mean tumor volume of the mice are shown in FIG. 2B.

As shown in FIG. 2A, mice treated with SEA-m1C10 (G3) significantly extended animal survival, as compared to that of the control group mice (G1). Further, concomitant administration of SEA-m1C10 and the anti-mPD1 surrogate antibody in a disseminated model of A20 did not exhibit any anti-tumor activity and even appeared antagonistic when administered together. In contrast, as shown in FIG. 2B, the anti-tumor activity was enhanced when SEAm1C10 was administered in a staggered manner with the anti-mPD-1 surrogate antibody. For example, while both the anti-mPD-1 surrogate antibody (G2) and SEA-m1C10 (G3) delayed tumor progression with 4/6 and ⅚ animals achieving a complete response (CR), respectively, the combinatorial activity of these two agents was striking, driving complete tumor regression in 6/6 animals (G4).

### RENCA Renal Cell Carcinoma Model

The combinatorial activity of the SEA-m1C10 antibody with the anti-mPD1 surrogate antibody was also tested in a subcutaneous RENCA renal cell syngeneic model. BALB/c mice were implanted with the RENCA syngeneic tumor cell line subcutaneously in the flank of mice on day 0. When the mean tumor size (measured using the formula: Volume (mm³) = 0.5 * Length * Width², wherein length is the longer dimension and width is the shorter dimension) reached 100 mm³, mice were randomly placed into a control group G1 and three treatment groups G2-G4 (5 mice per group).

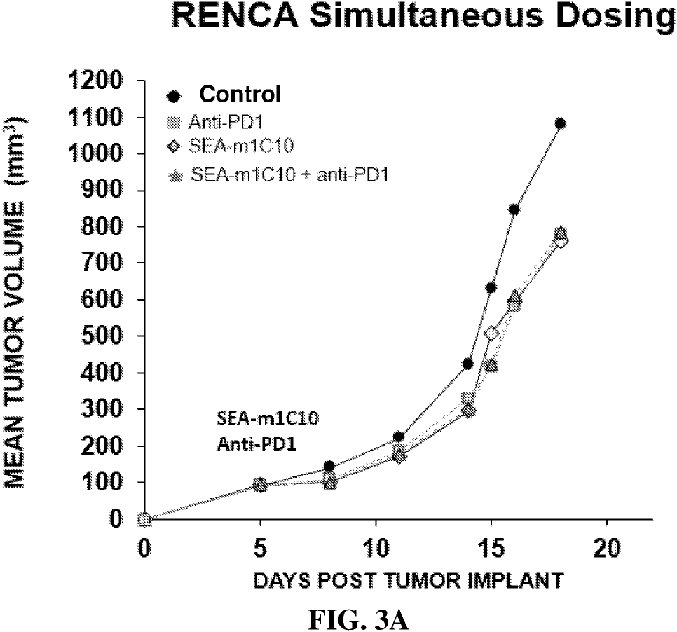

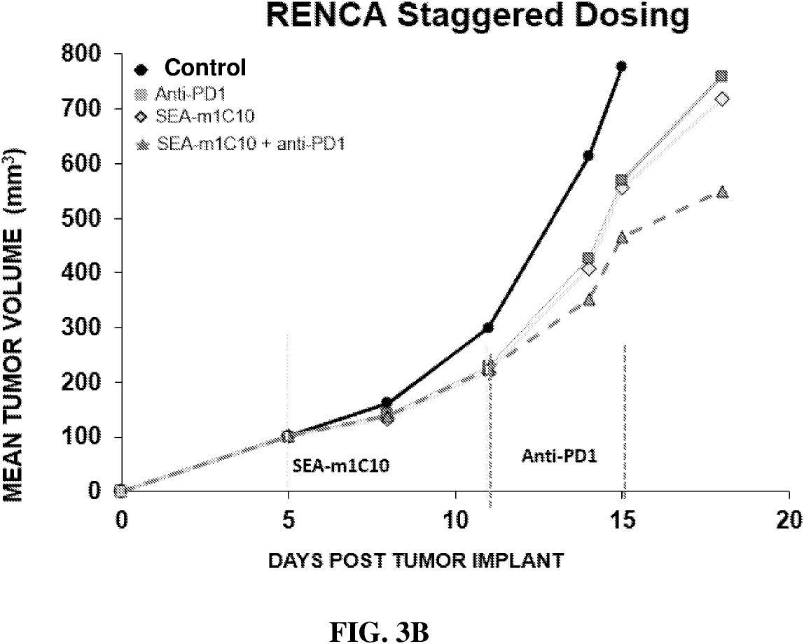

In one experiment, the treatment group mice were administered intraperitoneally with either a single agent (the anti-mPD-1 surrogate antibody (G2) or SEA-m1C10 (G3)), or a combination thereof (G4) on the same day. The administration frequency was once every three days for a total of three treatments. The control group mice (G1) were untreated. Mean tumor volume of the mice are shown in FIG. 3A.

Alternatively, mice in treatment groups G3 and G4 were administered with 3 doses of SEA-m1C10 three days apart (e.g., within the period from day 5 to day 11). On the last day of SEA-m1C10 treatment (e.g., on day 11), the first dose of the anti-mPD-1 surrogate antibody was administered to the G2 and G4 group mice, which then received 2 additional doses three days apart (e.g., within the period from day 9 to day 15). The control group mice (G1) were untreated. Mean tumor volume of the mice are shown in FIG. 3B.

As shown in FIG. 3A, the results showed that concomitant administration of SEAm1C10 and the anti-mPD-1 surrogate antibody did not exhibit any anti-tumor activity and even appeared antagonistic when administered together. As shown in FIG. 3B, while both SEAm1C10 and the anti-mPD-1 surrogate antibody delayed tumor progression, the combinatorial activity of these two agents administered in a staggered manner improved anti-tumor activity and resulted in tumor growth delay.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a pancreatic cancer comprising administering to a patient having the pancreatic cancer:
(i) a chemotherapy on day 1, day 8, and day 15 of each 28-day cycle,
(ii) a composition comprising an anti-CD40 antibody on day 3 of each 28-day cycle, and
(iii) an anti-PD-1 antibody on day 8 of each 42-day cycle;
wherein the anti-CD40 antibody: 1) comprises a heavy chain variable region comprising amino acid 1-113 of SEQ ID NO: 1 and a light chain variable region comprising amino acid 1-113 of SEQ ID NO: 2, and a human constant region; wherein the human constant region has an N-glycoside-linked sugar chain at residue N297 according to the EU index; and wherein less than 20% of N-glycoside-linked sugar chains in the composition comprise a fucose residue; and/or 2) is a SEA-CD40 variant; and
wherein the anti-PD-1 antibody comprises a light chain comprising CDRs of SEQ ID NOs: 3-5, and a heavy chain comprising CDRs of SEQ ID NOs 8-10.

2. The method of claim 1 wherein less than 10% of N-glycoside-linked sugar chains in the composition comprise a fucose residue.

3. The method of claim 1 or 2 wherein less than 5% of N-glycoside-linked sugar chains in the composition comprise a fucose residue.

4. The method of any one of claims 1-3 wherein less than 3% of N-glycoside-linked sugar chains in the composition comprise a fucose residue.

5. The method of any one of claims 1-4 wherein less than 2% of N-glycoside-linked sugar chains in the composition comprise a fucose residue.

6. The method of any one of claims 1-5 wherein the anti-CD40 antibody comprises a heavy chain comprising the amino acid sequence of SEQ ID NO: 1 and a light chain comprising the amino acid sequence of SEQ ID NO: 2.

7. The method of any one of claims 1-6, where in the anti-CD40 antibody is SEA-CD40.

8. The method of any one of claims 1-5 wherein the anti-CD40 antibody is a SEA-CD40 variant.

9. The method of any one of claims 1-8 wherein the light chain of the anti-PD-1 antibody has a light chain variable region comprising the amino acid sequence of SEQ ID NO: 6, and wherein the heavy chain of the anti-PD-1 antibody has a heavy chain variable region comprising the amino acid sequence of SEQ ID NO: 11.

10. The method of any one of claims 1-9 wherein the light chain of the anti-PD-1 antibody comprises the amino acid sequence of SEQ ID NO: 7, and wherein the heavy chain of the anti-PD-1 antibody comprises the amino acid sequence of SEQ ID NO: 12.

11. The method of any one of claims 1-10 wherein the anti-PD-1 antibody is pembrolizumab.

12. The method of any one of claims 1-9 wherein the anti-PD-1 antibody is a pembrolizumab variant.

13. The method of any one of claims 1-12 wherein the chemotherapy comprises gemcitabine and/or paclitaxel.

14. The method of claim 13 wherein paclitaxel is nab-paclitaxel.

15. The method of any one of claims 1-14 wherein the anti-CD40 antibody is administered at 10 µg/kg.

16. The method of any one of claims 1-14 wherein the anti-CD40 antibody is administered at 30 µg/kg.

17. The method of any one of claims 1-16 wherein the anti-PD-1 antibody is administered at 400 mg.

18. The method of any one of claims 1-17 wherein the anti-PD-1 antibody is administered intravenously.

19. The method of any one of claims 1-18, wherein the pancreatic cancer is pancreatic ductal adenocarcinoma (PDAC).

20. The method of any one of claims 1-19, wherein the anti-CD40 antibody is administered intravenously.

21. The method of any one of claims 1-19, wherein the anti-CD40 antibody is administered subcutaneously.

22. A method of treating a cancer comprising:
(i) administering a chemotherapy to a patient having the cancer in a cycle of every 4 weeks,
(ii) administering a composition comprising an anti-CD40 antibody to the patient in a cycle of every 4 weeks, and
(iii) administering an anti-PD-1 antibody to the patient in a cycle of every 3 weeks or 6 weeks,
wherein the chemotherapy is administered on day 1, day 8, day 15 of each 4-week cycle, the anti-CD40 antibody is administered on day 3 of each 4-week cycle, and the anti-PD-1 antibody is administered on day 8 of each of the 3-week cycle or 6-week cycle;
wherein the anti-CD40 antibody: 1) comprises a heavy chain variable region comprising amino acid 1-113 of SEQ ID NO: 1 and a light chain variable region comprising amino acid 1-113 of SEQ ID NO: 2, and a human constant region; wherein the human constant region has an N-glycoside-linked sugar chain at residue N297 according to the EU index; and wherein less than 20% of N-glycoside-linked sugar chains in the composition comprise a fucose residue; and/or 2) is a SEA-CD40 variant; and
wherein the anti-PD-1 antibody comprises a light chain comprising CDRs of SEQ ID NOs: 3-5, and a heavy chain comprising CDRs of SEQ ID NOs 8-10.

23. The method of claim 22 wherein less than 10% of N-glycoside-linked sugar chains in the composition comprise a fucose residue.

24. The method of claim 22 or 23 wherein less than 5% of N-glycoside-linked sugar chains in the composition comprise a fucose residue.

25. The method of any one of claims 22-24 wherein less than 3% of N-glycoside-linked sugar chains in the composition comprise a fucose residue.

26. The method of any one of claims 22-25 wherein less than 2% of N-glycoside-linked sugar chains in the composition comprise a fucose residue.

27. The method of any one of claims 22-26 wherein the anti-CD40 antibody comprises a heavy chain comprising the amino acid sequence of SEQ ID NO: 1 and a light chain comprising the amino acid sequence of SEQ ID NO: 2.

28. The method of any one of claims 22-27, where in the anti-CD40 antibody is SEA-CD40.

29. The method of any one of claims 22-26 wherein the anti-CD40 antibody is a SEA-CD40 variant.

30. The method of any one of claims 22-29 wherein the light chain of the anti-PD-1 antibody has a light chain variable region comprising the amino acid sequence of SEQ ID NO: 6, and wherein the heavy chain of the anti-PD-1 antibody has a heavy chain variable region comprising the amino acid sequence of SEQ ID NO: 11.

31. The method of any one of claims 22-30 wherein the light chain of the anti-PD-1 antibody comprises the amino acid sequence of SEQ ID NO: 7, and wherein the heavy chain of the anti-PD-1 antibody comprises the amino acid sequence of SEQ ID NO: 12.

32. The method of any one of claims 22-31 wherein the anti-PD-1 antibody is pembrolizumab.

33. The method of any one of claims 22-30 wherein the anti-PD-1 antibody is a pembrolizumab variant.

34. The method of any one of claims 22-33, wherein the anti-PD-1 antibody is administered in a cycle of every 3 weeks, and the anti-PD-1 antibody is administered on day 8 of each 3-week cycle at a dose of 200 mg.

35. The method of any one of claims 22-33, wherein the anti-PD-1 antibody is administered in a cycle of every 6 weeks, and the anti-PD-1 antibody is administered on day 8 of each 6-week cycle at a dose of 400 mg.

36. The method of any one of claims 22-35 wherein the anti-PD-1 antibody is administered intravenously.

37. A method of treating a cancer comprising:
(i) administering an anti-CD40 antibody to a patient having the cancer in a cycle of every week, every 2 weeks, every 3 weeks, every 4 weeks, every 5 weeks, every 6 weeks, every 7 weeks, or every 8 weeks, wherein the cycle comprises a first cycle of administration of the anti-CD40 antibody,
(ii) administering an anti-PD-1 antibody to the patient in a cycle of every 3 weeks or every 6 weeks, wherein the cycle comprises a first cycle of administration of the anti-PD-1 antibody,
wherein a first administration of the anti-CD40 antibody in the first cycle of administration of the anti-CD40 antibody is 1 day, 2 days, 3 days, 4 days, 5 days, 6 days or 7 days prior to a first administration of the anti-PD-1 antibody in the first cycle of administration of the anti-PD-1 antibody;
wherein the anti-CD40 antibody: 1) comprises a heavy chain variable region comprising amino acid 1-113 of SEQ ID NO: 1 and a light chain variable region comprising amino acid 1-113 of SEQ ID NO: 2, and a human constant region; wherein the human constant region has an N-glycoside-linked sugar chain at residue N297 according to the EU index; and wherein less than 20% of N-glycoside-linked sugar chains in the composition comprise a fucose residue; and/or 2) is a SEA-CD40 variant; and
wherein the anti-PD-1 antibody comprises a light chain comprising CDRs of SEQ ID NOs: 3-5, and a heavy chain comprising CDRs of SEQ ID NOs 8-10.

38. The method of claim 37 wherein less than 10% of N-glycoside-linked sugar chains in the composition comprise a fucose residue.

39. The method of claim 37 or 38 wherein less than 5% of N-glycoside-linked sugar chains in the composition comprise a fucose residue.

40. The method of any one of claims 37-39 wherein less than 3% of N-glycoside-linked sugar chains in the composition comprise a fucose residue.

41. The method of any one of claims 37-40 wherein less than 2% of N-glycoside-linked sugar chains in the composition comprise a fucose residue.

42. The method of any one of claims 37-41 wherein the anti-CD40 antibody comprises a heavy chain comprising the amino acid sequence of SEQ ID NO: 1 and a light chain comprising the amino acid sequence of SEQ ID NO: 2.

43. The method of any one of claims 37-42, where in the anti-CD40 antibody is SEA-CD40.

44. The method of any one of claims 37-41 wherein the anti-CD40 antibody is a SEA-CD40 variant.

45. The method of any one of claims 37-44 wherein the light chain of the anti-PD-1 antibody has a light chain variable region comprising the amino acid sequence of SEQ ID NO: 6, and wherein the heavy chain of the anti-PD-1 antibody has a heavy chain variable region comprising the amino acid sequence of SEQ ID NO: 11.

46. The method of any one of claims 37-45 wherein the light chain of the anti-PD-1 antibody comprises the amino acid sequence of SEQ ID NO: 7, and wherein the heavy chain of the anti-PD-1 antibody comprises the amino acid sequence of SEQ ID NO: 12.

47. The method of any one of claims 37-46 wherein the anti-PD-1 antibody is pembrolizumab.

48. The method of any one of claims 37-45 wherein the anti-PD-1 antibody is a pembrolizumab variant.

49. The method of any one of claims 37-48, wherein the anti-CD40 antibody is administered in a cycle of every 2 weeks, every 4 weeks, every 6 weeks, or every 8 weeks.

50. The method of any one of claims 37-49, wherein the anti-CD40 antibody is administered in a cycle of every 4 weeks or every 8 weeks.

51. The method of any one of claims 37-50, wherein the anti-CD40 antibody is administered in a cycle of every 4 weeks.

52. The method of any one of claims 37-51, wherein the anti-PD-1 antibody is administered in a cycle of every 3 weeks at a dose of 200 mg.

53. The method of any one of claims 37-51, wherein the anti-PD-1 antibody is administered in a cycle of every 6 weeks at a dose of 400 mg.

54. The method of any one of claims 37-53 wherein the anti-PD-1 antibody is administered intravenously.

55. The method of any one of claims 37-54, wherein the first administration of the anti-CD40 antibody in the first cycle is 2 days, 3 days, 4 days, 5 days, or 6 days prior to the first administration of the anti-PD-1 antibody in the first cycle.

56. The method of any one of claims 37-55, wherein the first administration of the anti-CD40 antibody in the first cycle is 3 days, 4 days, or 5 days prior to the first administration of the anti-PD-1 antibody in the first cycle.

57. The method of any one of claims 37-56, wherein the first administration of the anti-CD40 antibody in the first cycle is 5 days prior to the first administration of the anti-PD-1 antibody in the first cycle.

58. The method of any one of claims 37-48, wherein the anti-CD40 antibody and the anti-PD-1 antibody are administered in their first cycles according to a treatment regimen selected from the group consisting of:
the anti-CD40 antibody is first administered on day 1, and the anti-PD-1 antibody is first administered on day 2;
the anti-CD40 antibody is first administered on day 1, and the anti-PD-1 antibody is first administered on day 3;
the anti-CD40 antibody is first administered on day 1, and the anti-PD-1 antibody is first administered on day 4;
the anti-CD40 antibody is first administered on day 1, and the anti-PD-1 antibody is first administered on day 5;
the anti-CD40 antibody is first administered on day 1, and the anti-PD-1 antibody is first administered on day 6;
the anti-CD40 antibody is first administered on day 1, and the anti-PD-1 antibody is first administered on day 7;
the anti-CD40 antibody is first administered on day 1, and the anti-PD-1 antibody is first administered on day 8;
the anti-CD40 antibody is first administered on day 2, and the anti-PD-1 antibody is first administered on day 3;
the anti-CD40 antibody is first administered on day 2, and the anti-PD-1 antibody is first administered on day 4;
the anti-CD40 antibody is first administered on day 2, and the anti-PD-1 antibody is first administered on day 5;
the anti-CD40 antibody is first administered on day 2, and the anti-PD-1 antibody is first administered on day 6;
the anti-CD40 antibody is first administered on day 2, and the anti-PD-1 antibody is first administered on day 7;
the anti-CD40 antibody is first administered on day 2, and the anti-PD-1 antibody is first administered on day 8;
the anti-CD40 antibody is first administered on day 3, and the anti-PD-1 antibody is first administered on day 4;
the anti-CD40 antibody is first administered on day 3, and the anti-PD-1 antibody is first administered on day 5;
the anti-CD40 antibody is first administered on day 3, and the anti-PD-1 antibody is first administered on day 6;
the anti-CD40 antibody is first administered on day 3, and the anti-PD-1 antibody is first administered on day 7;
the anti-CD40 antibody is first administered on day 3, and the anti-PD-1 antibody is first administered on day 8;
the anti-CD40 antibody is first administered on day 4, and the anti-PD-1 antibody is first administered on day 5;
the anti-CD40 antibody is first administered on day 4, and the anti-PD-1 antibody is first administered on day 6;
the anti-CD40 antibody is first administered on day 4, and the anti-PD-1 antibody is first administered on day 7;
the anti-CD40 antibody is first administered on day 4, and the anti-PD-1 antibody is first administered on day 8;
the anti-CD40 antibody is first administered on day 5, and the anti-PD-1 antibody is first administered on day 6;
the anti-CD40 antibody is first administered on day 5, and the anti-PD-1 antibody is first administered on day 7;
the anti-CD40 antibody is first administered on day 5, and the anti-PD-1 antibody is first administered on day 8;
the anti-CD40 antibody is first administered on day 6, and the anti-PD-1 antibody is first administered on day 7;
the anti-CD40 antibody is first administered on day 6, and the anti-PD-1 antibody is first administered on day 8; and
the anti-CD40 antibody is first administered on day 7, and the anti-PD-1 antibody is first administered on day 8.

59. The method of claim 58, wherein the anti-CD40 antibody is first administered on day 1, and the anti-PD-1 antibody is first administered on day 3.

60. The method of claim 58, wherein the anti-CD40 antibody is first administered on day 1, and the anti-PD-1 antibody is first administered on day 5.

61. The method of claim 58, wherein the anti-CD40 antibody is first administered on day 1, and the anti-PD-1 antibody is first administered on day 8.

62. The method of claim 58, wherein the anti-CD40 antibody is first administered on day 3, and the anti-PD-1 antibody is first administered on day 5.

63. The method of claim 58, wherein the anti-CD40 antibody is first administered on day 3, and the anti-PD-1 antibody is first administered on day 8.

64. The method of claim 58, wherein the anti-CD40 antibody is first administered on day 5, and the anti-PD-1 antibody is first administered on day 8.

65. A method of treating a cancer comprising:
(i) administering a chemotherapy to a patient having the cancer in a cycle of every 3 weeks, every 4 weeks, every 5 weeks, or every 6 weeks,
(ii) administering an anti-CD40 antibody to a patient having the cancer in a cycle of every week, every 2 weeks, every 3 weeks, every 4 weeks, every 5 weeks, every 6 weeks, every 7 weeks, or every 8 weeks, and
(iii) administering an anti-PD-1 antibody to the patient in a cycle of every 3 weeks or every 6 weeks,
wherein a first administration of the chemotherapy in the first cycle of administration of the chemotherapy is 1 day, 2 days, 3 days, 4 days, 5 days, 6 days or 7 days prior to a first administration of the anti-CD40 antibody in the first cycle of administration of the anti-CD40 antibody,
wherein a first administration of the anti-CD40 antibody in the first cycle of administration of the anti-CD40 antibody is 1 day, 2 days, 3 days, 4 days, 5 days, 6 days or 7 days prior to the first administration of the anti-PD-1 antibody in the first cycle of administration of the anti-PD-1 antibody;
wherein the anti-CD40 antibody: 1) comprises a heavy chain variable region comprising amino acid 1-113 of SEQ ID NO: 1 and a light chain variable region comprising amino acid 1-113 of SEQ ID NO: 2, and a human constant region; wherein the human constant region has an N-glycoside-linked sugar chain at residue N297 according to the EU index; and wherein less than 20% of N-glycoside-linked sugar chains in the composition comprise a fucose residue; and/or 2) is a SEA-CD40 variant; and
wherein the anti-PD-1 antibody comprises a light chain comprising CDRs of SEQ ID NOs: 3-5, and a heavy chain comprising CDRs of SEQ ID NOs 8-10.

66. The method of claim 65 wherein less than 10% of N-glycoside-linked sugar chains in the composition comprise a fucose residue.

67. The method of claim 65 or 66 wherein less than 5% of N-glycoside-linked sugar chains in the composition comprise a fucose residue.

68. The method of any one of claims 65-67 wherein less than 3% of N-glycoside-linked sugar chains in the composition comprise a fucose residue.

69. The method of any one of claims 65-68 wherein less than 2% of N-glycoside-linked sugar chains in the composition comprise a fucose residue.

70. The method of any one of claims 65-69 wherein the anti-CD40 antibody comprises a heavy chain comprising the amino acid sequence of SEQ ID NO: 1 and a light chain comprising the amino acid sequence of SEQ ID NO: 2.

71. The method of any one of claims 65-70, where in the anti-CD40 antibody is SEA-CD40.

72. The method of any one of claims 65-69 wherein the anti-CD40 antibody is a SEA-CD40 variant.

73. The method of any one of claims 65-72 wherein the light chain of the anti-PD-1 antibody has a light chain variable region comprising the amino acid sequence of SEQ ID NO: 6, and wherein the heavy chain of the anti-PD-1 antibody has a heavy chain variable region comprising the amino acid sequence of SEQ ID NO: 11.

74. The method of any one of claims 65-73 wherein the light chain of the anti-PD-1 antibody comprises the amino acid sequence of SEQ ID NO: 7, and wherein the heavy chain of the anti-PD-1 antibody comprises the amino acid sequence of SEQ ID NO: 12.

75. The method of any one of claims 65-74 wherein the anti-PD-1 antibody is pembrolizumab.

76. The method of any one of claims 65-73 wherein the anti-PD-1 antibody is a pembrolizumab variant.

77. The method of any one of claims 65-76, wherein the chemotherapy comprises one or both of gemcitabine and paclitaxel.

78. The method of any one of claims 65-77, wherein the chemotherapy comprises both gemcitabine and paclitaxel.

79. The method of any one of claims 65-78, wherein the chemotherapy consists of gemcitabine and paclitaxel.

80. The method of any one of claims 77-79, wherein paclitaxel is nab-paclitaxel.

81. The method of any one of claims 77-79, wherein paclitaxel is albumin-bound paclitaxel.

82. The method of any one of claims 65-81, wherein the anti-CD40 antibody is administered in a cycle of every 2 weeks, every 4 weeks, every 6 weeks, or every 8 weeks.

83. The method of any one of claims 65-82, wherein the anti-CD40 antibody is administered in a cycle of every 4 weeks or every 8 weeks.

84. The method of any one of claims 65-83, wherein the anti-CD40 antibody is administered in a cycle of every 4 weeks.

85. The method of any one of claims 65-84, wherein the anti-PD-1 antibody is administered in a cycle of every 3 weeks at a dose of 200 mg.

86. The method of any one of claims 65-85, wherein the anti-PD-1 antibody is administered in a cycle of every 6 weeks at a dose of 400 mg.

87. The method of any one of claims 65-86 wherein the anti-PD-1 antibody is administered intravenously.

88. The method of any one of claims 65-87, wherein the first administration of the chemotherapy in the first cycle is 2 days, 3 days, 4 days, 5 days, or 6 days prior to the first administration of the anti-CD40 antibody in the first cycle, and
wherein the first administration of the anti-CD40 antibody in the first cycle is 2 days, 3 days, 4 days, 5 days, or 6 days prior to the first administration of the anti-PD-1 antibody in the first cycle.

89. The method of any one of claims 65-88, wherein the first administration of the chemotherapy in the first cycle is 2 days, 3 days, or 4 days prior to the first administration of the anti-CD40 antibody in the first cycle, and
wherein the first administration of the anti-CD40 antibody in the first cycle is 3 days, 4 days, or 5 days prior to the first administration of the anti-PD-1 antibody in the first cycle.

90. The method of any one of claims 33-45, where the first administration of the chemotherapy in the first cycle is 2 days prior to the first administration of the anti-CD40 antibody in the first cycle, and
wherein the first administration of the anti-CD40 antibody in the first cycle is 5 days prior to the first administration of the anti-PD-1 antibody in the first cycle.

91. The method of any one of claims 65-87, wherein the chemotherapy, the anti-CD40 antibody and the anti-PD-1 antibody are administered in their first cycles according to a treatment regimen selected from the group consisting of:
the chemotherapy is first administered on day 1, the anti-CD40 antibody is first administered on day 2, and the anti-PD-1 antibody is first administered on day 3;
the chemotherapy is first administered on day 1, the anti-CD40 antibody is first administered on day 2, and the anti-PD-1 antibody is first administered on day 4;
the chemotherapy is first administered on day 1, the anti-CD40 antibody is first administered on day 2, and the anti-PD-1 antibody is first administered on day 5;
the chemotherapy is first administered on day 1, the anti-CD40 antibody is first administered on day 2, and the anti-PD-1 antibody is first administered on day 6;
the chemotherapy is first administered on day 1, the anti-CD40 antibody is first administered on day 2, and the anti-PD-1 antibody is first administered on day 7;
the chemotherapy is first administered on day 1, the anti-CD40 antibody is first administered on day 2, and the anti-PD-1 antibody is first administered on day 8;
the chemotherapy is first administered on day 1, the anti-CD40 antibody is first administered on day 2, and the anti-PD-1 antibody is first administered on day 9;
the chemotherapy is first administered on day 1, the anti-CD40 antibody is first administered on day 2, and the anti-PD-1 antibody is first administered on day 10;
the chemotherapy is first administered on day 1, the anti-CD40 antibody is first administered on day 2, and the anti-PD-1 antibody is first administered on day 11;
the chemotherapy is first administered on day 1, the anti-CD40 antibody is first administered on day 2, and the anti-PD-1 antibody is first administered on day 12;
the chemotherapy is first administered on day 1, the anti-CD40 antibody is first administered on day 2, and the anti-PD-1 antibody is first administered on day 13;
the chemotherapy is first administered on day 1, the anti-CD40 antibody is first administered on day 2, and the anti-PD-1 antibody is first administered on day 14;
the chemotherapy is first administered on day 1, the anti-CD40 antibody is first administered on day 2, and the anti-PD-1 antibody is first administered on day 15;
the chemotherapy is first administered on day 1, the anti-CD40 antibody is first administered on day 3, and the anti-PD-1 antibody is first administered on day 4;
the chemotherapy is first administered on day 1, the anti-CD40 antibody is first administered on day 3, and the anti-PD-1 antibody is first administered on day 5;
the chemotherapy is first administered on day 1, the anti-CD40 antibody is first administered on day 3, and the anti-PD-1 antibody is first administered on day 6;
the chemotherapy is first administered on day 1, the anti-CD40 antibody is first administered on day 3, and the anti-PD-1 antibody is first administered on day 7;
the chemotherapy is first administered on day 1, the anti-CD40 antibody is first administered on day 3, and the anti-PD-1 antibody is first administered on day 8;
the chemotherapy is first administered on day 1, the anti-CD40 antibody is first administered on day 3, and the anti-PD-1 antibody is first administered on day 9;
the chemotherapy is first administered on day 1, the anti-CD40 antibody is first administered on day 3, and the anti-PD-1 antibody is first administered on day 10;
the chemotherapy is first administered on day 1, the anti-CD40 antibody is first administered on day 3, and the anti-PD-1 antibody is first administered on day 11;
the chemotherapy is first administered on day 1, the anti-CD40 antibody is first administered on day 3, and the anti-PD-1 antibody is first administered on day 12;
the chemotherapy is first administered on day 1, the anti-CD40 antibody is first administered on day 3, and the anti-PD-1 antibody is first administered on day 13;
the chemotherapy is first administered on day 1, the anti-CD40 antibody is first administered on day 3, and the anti-PD-1 antibody is first administered on day 14;
the chemotherapy is first administered on day 1, the anti-CD40 antibody is first administered on day 3, and the anti-PD-1 antibody is first administered on day 15;
the chemotherapy is first administered on day 1, the anti-CD40 antibody is first administered on day 4, and the anti-PD-1 antibody is first administered on day 5;
the chemotherapy is first administered on day 1, the anti-CD40 antibody is first administered on day 4, and the anti-PD-1 antibody is first administered on day 6;
the chemotherapy is first administered on day 1, the anti-CD40 antibody is first administered on day 4, and the anti-PD-1 antibody is first administered on day 7;
the chemotherapy is first administered on day 1, the anti-CD40 antibody is first administered on day 4, and the anti-PD-1 antibody is first administered on day 8;
the chemotherapy is first administered on day 1, the anti-CD40 antibody is first administered on day 4, and the anti-PD-1 antibody is first administered on day 9;
the chemotherapy is first administered on day 1, the anti-CD40 antibody is first administered on day 4, and the anti-PD-1 antibody is first administered on day 10;
the chemotherapy is first administered on day 1, the anti-CD40 antibody is first administered on day 4, and the anti-PD-1 antibody is first administered on day 11;
the chemotherapy is first administered on day 1, the anti-CD40 antibody is first administered on day 4, and the anti-PD-1 antibody is first administered on day 12;
the chemotherapy is first administered on day 1, the anti-CD40 antibody is first administered on day 4, and the anti-PD-1 antibody is first administered on day 13;
the chemotherapy is first administered on day 1, the anti-CD40 antibody is first administered on day 4, and the anti-PD-1 antibody is first administered on day 14;
the chemotherapy is first administered on day 1, the anti-CD40 antibody is first administered on day 4, and the anti-PD-1 antibody is first administered on day 15;
the chemotherapy is first administered on day 1, the anti-CD40 antibody is first administered on day 5, and the anti-PD-1 antibody is first administered on day 6;
the chemotherapy is first administered on day 1, the anti-CD40 antibody is first administered on day 5, and the anti-PD-1 antibody is first administered on day 7;
the chemotherapy is first administered on day 1, the anti-CD40 antibody is first administered on day 5, and the anti-PD-1 antibody is first administered on day 8;
the chemotherapy is first administered on day 1, the anti-CD40 antibody is first administered on day 5, and the anti-PD-1 antibody is first administered on day 9;
the chemotherapy is first administered on day 1, the anti-CD40 antibody is first administered on day 5, and the anti-PD-1 antibody is first administered on day 10;
the chemotherapy is first administered on day 1, the anti-CD40 antibody is first administered on day 5, and the anti-PD-1 antibody is first administered on day 11;
the chemotherapy is first administered on day 1, the anti-CD40 antibody is first administered on day 5, and the anti-PD-1 antibody is first administered on day 12;
the chemotherapy is first administered on day 1, the anti-CD40 antibody is first administered on day 5, and the anti-PD-1 antibody is first administered on day 13;
the chemotherapy is first administered on day 1, the anti-CD40 antibody is first administered on day 5, and the anti-PD-1 antibody is first administered on day 14;
the chemotherapy is first administered on day 1, the anti-CD40 antibody is first administered on day 5, and the anti-PD-1 antibody is first administered on day 15;
the chemotherapy is first administered on day 1, the anti-CD40 antibody is first administered on day 6, and the anti-PD-1 antibody is first administered on day 7;
the chemotherapy is first administered on day 1, the anti-CD40 antibody is first administered on day 6, and the anti-PD-1 antibody is first administered on day 8;
the chemotherapy is first administered on day 1, the anti-CD40 antibody is first administered on day 6, and the anti-PD-1 antibody is first administered on day 9;
the chemotherapy is first administered on day 1, the anti-CD40 antibody is first administered on day 6, and the anti-PD-1 antibody is first administered on day 10;
the chemotherapy is first administered on day 1, the anti-CD40 antibody is first administered on day 6, and the anti-PD-1 antibody is first administered on day 11;
the chemotherapy is first administered on day 1, the anti-CD40 antibody is first administered on day 6, and the anti-PD-1 antibody is first administered on day 11;
the chemotherapy is first administered on day 1, the anti-CD40 antibody is first administered on day 6, and the anti-PD-1 antibody is first administered on day 13;
the chemotherapy is first administered on day 1, the anti-CD40 antibody is first administered on day 6, and the anti-PD-1 antibody is first administered on day 14;
the chemotherapy is first administered on day 1, the anti-CD40 antibody is first administered on day 6, and the anti-PD-1 antibody is first administered on day 15;
the chemotherapy is first administered on day 1, the anti-CD40 antibody is first administered on day 7, and the anti-PD-1 antibody is first administered on day 8;
the chemotherapy is first administered on day 1, the anti-CD40 antibody is first administered on day 7, and the anti-PD-1 antibody is first administered on day 9;
the chemotherapy is first administered on day 1, the anti-CD40 antibody is first administered on day 7, and the anti-PD-1 antibody is first administered on day 10;
the chemotherapy is first administered on day 1, the anti-CD40 antibody is first administered on day 7, and the anti-PD-1 antibody is first administered on day 11;
the chemotherapy is first administered on day 1, the anti-CD40 antibody is first administered on day 7, and the anti-PD-1 antibody is first administered on day 12;
the chemotherapy is first administered on day 1, the anti-CD40 antibody is first administered on day 7, and the anti-PD-1 antibody is first administered on day 13;
the chemotherapy is first administered on day 1, the anti-CD40 antibody is first administered on day 7, and the anti-PD-1 antibody is first administered on day 14;
the chemotherapy is first administered on day 1, the anti-CD40 antibody is first administered on day 7, and the anti-PD-1 antibody is first administered on day 15;
the chemotherapy is first administered on day 1, the anti-CD40 antibody is first administered on day 8, and the anti-PD-1 antibody is first administered on day 9;
the chemotherapy is first administered on day 1, the anti-CD40 antibody is first administered on day 8, and the anti-PD-1 antibody is first administered on day 10;
the chemotherapy is first administered on day 1, the anti-CD40 antibody is first administered on day 8, and the anti-PD-1 antibody is first administered on day 11;
the chemotherapy is first administered on day 1, the anti-CD40 antibody is first administered on day 8, and the anti-PD-1 antibody is first administered on day 12;
the chemotherapy is first administered on day 1, the anti-CD40 antibody is first administered on day 8, and the anti-PD-1 antibody is first administered on day 13;
the chemotherapy is first administered on day 1, the anti-CD40 antibody is first administered on day 8, and the anti-PD-1 antibody is first administered on day 14; and
the chemotherapy is first administered on day 1, the anti-CD40 antibody is first administered on day 8, and the anti-PD-1 antibody is first administered on day 15.

92. The method of claim 91 wherein the chemotherapy is first administered on day 1, the anti-CD40 antibody is first administered on day 3, and the anti-PD-1 antibody is first administered on day 8.

93. The method of claim 91 wherein the chemotherapy is first administered on day 1, the anti-CD40 antibody is first administered on day 5, and the anti-PD-1 antibody is first administered on day 8.

94. The method of claim 91 wherein the chemotherapy is first administered on day 1, the anti-CD40 antibody is first administered on day 7, and the anti-PD-1 antibody is first administered on day 8.

95. The method of claim 91 wherein the chemotherapy is first administered on day 1, the anti-CD40 antibody is first administered on day 7, and the anti-PD-1 antibody is first administered on day 15.

96. The method of claim 91 wherein the chemotherapy is first administered on day 1, the anti-CD40 antibody is first administered on day 8, and the anti-PD-1 antibody is first administered on day 10, day 11, day 12, or day 15.

97. The method of claim 91 wherein the chemotherapy is first administered on day 1, the anti-CD40 antibody is first administered on day 8, and the anti-PD-1 antibody is first administered on day 15.

98. The method of any one of claims 65-97, wherein the chemotherapy is administered in a cycle of every 4 weeks.

99. The method of any one of claims 65-97, wherein the chemotherapy is administered on day 1, day 5, and day 8 of each cycle.

100. The method of any one of claims 65-99, wherein the anti-CD40 antibody is administered in a cycle of every 4 weeks.

101. The method of any one of claims 22-100, wherein the anti-CD40 antibody is administered at a dose of about 3 µg/kg, about 10 µg/kg, about 30 µg/kg, about 45 µg/kg, or about 60 µg/kg patient body weight.

102. The method of claim 101, wherein the anti-CD40 antibody is administered at a dose of about 10 µg/kg patient body weight.

103. The method of claim 101, wherein the anti-CD40 antibody is administered at a dose of about 30 µg/kg patient body weight.

104. The method of any one of claims 22-103, wherein the cancer is melanoma; breast cancer, metastatic breast cancer; lung cancer, non-small cell lung cancer (NSCLC), or pancreatic cancer.

105. The method of any one of claims 22-104, wherein the cancer is pancreatic cancer.

106. The method of any one of claims 22-105, wherein the cancer is pancreatic ductal adenocarcinoma (PDAC).

107. The method of any one of claims 22-106, wherein the cancer is metastatic pancreatic ductal adenocarcinoma.
